(19) United States
(12) Patent Application Publication
(10) Pub. No.: US 2026/0034098 A1
(43) Pub. Date: Feb. 5, 2026

(54) BRIDGED TRICYCLIC GABA DERIVATIVES AS CALCIUM CHANNEL MODULATORS, METHODS OF MAKING AND METHODS OF USING THEREOF

(71) Applicant: Humanwell Pharmaceutical US, Inc., Ballwin, MO (US)

(72) Inventors: Hariprasada Reddy Kanna Reddy, Ballwin, MO (US); Shivansh Kaushik, Ballwin, MO (US); Shuo Zhao, Ballwin, MO (US); Ming Li, Ballwin, MO (US); Haizhou Sun, Ballwin, MO (US); Subo Liao, Ballwin, MO (US); Jun Yang, Ballwin, MO (US); Mingshu Wang, Ballwin, MO (US); Hao Zhou, Yichang (CN); Qing Xiong, Ballwin, MO (US); Xiongjun Mou, Yichang (CN)

(21) Appl. No.: 19/247,107

(22) Filed: Jun. 24, 2025

Related U.S. Application Data
(60) Provisional application No. 63/663,465, filed on Jun. 24, 2024.

(51) Int. Cl. A61K 31/41; A61K 31/197; A61K 31/215; A61P 25/04; C07B 59/00; C07C 69/608; C07C 211/41; C07D 257/04

(52) U.S. Cl. CPC A61K 31/41; A61K 31/197; A61K 31/215; A61P 25/04; C07B 59/001; C07B 59/002; C07C 69/608; C07C 211/41; C07D 257/04; C07B 2200/05; C07C 2603/66

(57) ABSTRACT

This invention discloses bridged tricyclic γ-aminobutyric acid (GABA) derivatives targeting the α2δ subunit of voltage-gated calcium channels. These compounds exhibit enhanced binding affinity and improved pharmacological profiles. They aim to overcome limitations of current α2δ ligands like gabapentin and pregabalin. The new derivatives show potential for treating neuropathic pain, epilepsy, and related disorders.

### CROSS REFERENCE TO RELATED APPLICATION

This application claims priority to U.S. Provisional Patent Application Ser. No. 63/663,465, filed Jun. 24, 2024, the disclosure of which is hereby incorporated by reference in their entirety.

### FIELD OF THE INVENTION

The present invention relates to newly designed bridged tricyclic γ-aminobutyric acid (GABA) derivatives that act as ligands targeting the α2δ subunit of voltage-gated calcium channels (VGCCs). These molecules exhibit promising pharmacological profiles and potential therapeutic utility.

### BACKGROUND OF THE INVENTION

Voltage-gated calcium channels (VGCCs) are integral membrane proteins that regulate calcium influx into excitable cells. They are broadly classified into high-voltage-activated (HVA) and low-voltage-activated (LVA) types. HVA channels consist of a pore-forming α1 subunit, alongside auxiliary α2δ, β, and γ subunits. [Catterall, Annual. Rev. Cell Dev. Biol. 2000, 16, 521-555]. Among them, the α2δ subunit is crucial for modulating the trafficking, membrane density, and biophysical properties of the channel complex [Felix et al., J. Neurosci. 1997, 17, 6884-6891; Klugbauer et al., J. Neurosci. 1999, 19, 684-691; Hobometal., Eur. J. Neurosci. 2000, 12, 1217-1226; and Qin et al., Mol. Pharmacol. 2002, 62, 485-496]. It has been well-documented that this subunit undergoes extensive glycosylation and is essential for channel maturation and surface expression [Sandoval et al., FBBS Lett. 2004, 576, 21-26; and Tetreault et al., J. Bio. Chem. 2016, 291, 4826-4843].

Pharmacological agents such as gabapentin, pregabalin, mirogabalin, and crisugabalin demonstrate strong affinity for the α2δ subunit, contributing to their therapeutic efficacy. These compounds are known to influence calcium signaling by modulating the interaction between VGCCs and N-methyl-D-aspartate (NMDA) receptors. Clinically, they are employed to manage neuropathic pain resulting from nerve injury, and have shown benefit in conditions such as epilepsy, diabetic peripheral neuropathy, and postherpetic neuralgia [Thorpe et al., comprehensive medicinal chemistry II, 2008, 8, 227-246]. Furthermore, they have been used off-label for a range of disorders including anxiety, depression, bipolar disorder, chronic pain syndromes, insomnia, and restless legs syndrome [Mack et al., J Manag Care Pharm 2003, 9, 559-568; and González-Bueno et al., Eur J Hosp Pharm 2015, 22, A84-A85].

Despite their clinical utility, existing α2δ ligands like gabapentin and pregabalin exhibit limitations in efficacy—typically showing only around 50-60% effectiveness in certain neuropathic pain indications such as diabetic peripheral neuropathy and postherpetic neuralgia. Moreover, they are associated with central nervous system (CNS) adverse effects, such as dizziness and somnolence. These drawbacks highlight the unmet need for next-generation compounds with improved therapeutic performance, enhanced safety profiles, and broader applicability in neuropathic pain and related neurological disorders.

### SUMMARY OF THE INVENTION

This summary is not intended to limit the scope of the claims.

The present disclosure is directed to compounds of formula (I), their stereoisomers, or a pharmaceutically acceptable salt thereof,

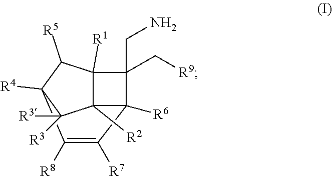

R1, R2, R3, R3′, R4, R5, R6, R7, and R8 are each independently selected from hydrogen, deuterium, halogen, substituted or unsubstituted C1-C6 alkyl, and substituted or unsubstituted C3-C6 cycloalkyl, wherein the substituted C1-C6 alkyl and substituted C3-C6 cycloalkyl are optionally further substituted with 0 to 6 substituents selected from halogen, hydroxyl, carboxyl, amino, cyano, C1-C3 alkyl, and C3-C6 cycloalkyl; and R9 is selected from carboxylic acid (—COOH),

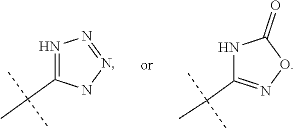

The present disclosure is directed to an intermediate, its stereoisomer, or a pharmaceutically acceptable salt thereof, for the preparation of the compound of general formula (I), wherein:

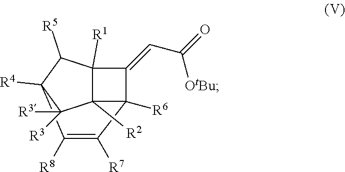

R1, R2, R3, R3′, R4, R5, R6, R7, and R8 are each independently selected from hydrogen, deuterium, halogen, substituted or unsubstituted C1-C6 alkyl, and substituted or unsubstituted C3-C6 cycloalkyl, wherein the substituted C1-C6 alkyl and substituted C3-C6 cycloalkyl are optionally further substituted with 0 to 6 substituents selected from halogen, hydroxyl, carboxyl, amino, cyano, C1-C3 alkyl, and C3-C6 cycloalkyl.

The present disclosure is further directed a pharmaceutical composition comprising a compound of formula (I), or a stereoisomer, solvate, metabolite, prodrug, pharmaceutically acceptable salt, or cocrystal thereof, and one or more pharmaceutically acceptable carriers and/or excipients.

The present disclosure is further directed to a method of treating and/or preventing diseases or disorders associated with ligands of the α2δ subunit of voltage-gated calcium channels in a subject in need thereof. The method comprises administering a therapeutically effective dose of the compound of formula (I).

The present disclosure is further directed to a method of treating pain mediated by the α2δ subunit of a voltage-gated calcium channel in a subject in need thereof.

### DETAILED DESCRIPTION

The following provides an overview of the subject matter described in detail herein.

The present invention relates to a novel class of compounds that function as ligands for the α2δ subunit of voltage-gated calcium channels, as well as methods for their preparation and their therapeutic applications. The compounds described herein can provide effective treatment for pain.

In one aspect, the invention provides a compound of formula (I), a stereoisomer thereof, or a pharmaceutically acceptable salt thereof:

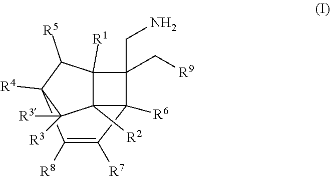

R1, R2, R3, R3′, R4, R5, R6, R7, and R8 are each independently selected from hydrogen, deuterium, halogen, substituted or unsubstituted C1-C6 alkyl, and substituted or unsubstituted C3-C6 cycloalkyl, wherein the substituted C1-C6 alkyl and substituted C3-C6 cycloalkyl are optionally further substituted with 1 to 6 substituents selected from halogen, hydroxyl, carboxyl, amino, cyano, C1-C3 alkyl, and C3-C6 cycloalkyl; and
R9 is selected from carboxylic acid (—COOH),

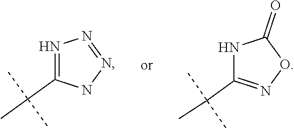

Specifically, in certain embodiments, the present invention provides a compound of formula (I), a stereoisomer thereof, or a pharmaceutically acceptable salt thereof:
wherein,
R1, R2, R3, R3′, R4, R5, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl;
R9 is selected from carboxylic acid (—COOH),

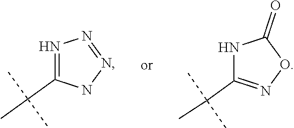

More specifically, in certain embodiments, the compound is of formula (II), or a stereoisomer or a pharmaceutically acceptable salt thereof:

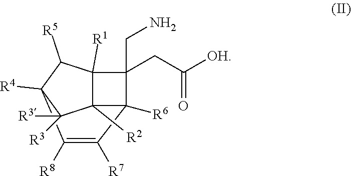

R1 and R5 are each independently selected from hydrogen, deuterium, methyl, ethyl, propyl, isopropyl, butyl, tert-butyl, and substituted C1-C6 alkyl, wherein the substituted C1-C6 alkyl is optionally further substituted with 1 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

R4, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

More preferably, in certain specific embodiments, the compound is of formula (II-1), or a stereoisomer or a pharmaceutically acceptable salt thereof,

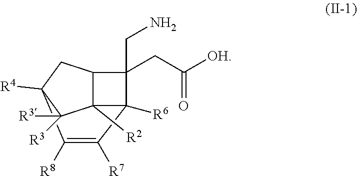

R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

R4, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

In certain embodiments, R2 is selected from hydrogen, deuterium, fluorine, chlorine, bromine, iodine, methyl, ethyl, propyl, isopropyl, butyl, tert-butyl, and substituted C1-C6 alkyl, wherein the substituted C1-C6 alkyl is optionally further substituted with 1 to 6 substituents selected from deuterium, fluorine, chlorine, bromine, iodine, hydroxyl, carboxyl, amino, cyano, methyl, ethyl, propyl, and isopropyl.

In certain embodiments, R3 and R3′ are each independently selected from hydrogen, deuterium, fluorine, chlorine, bromine, iodine, methyl, ethyl, propyl, isopropyl, butyl, tert-butyl, and substituted C1-C6 alkyl, wherein the substituted C1-C6 alkyl is optionally further substituted with 1 to 6 substituents selected from deuterium, fluorine, chlorine, bromine, iodine, hydroxyl, carboxyl, amino, cyano, methyl, ethyl, propyl, and isopropyl.

In certain embodiments, R4 and R6 are each independently selected from hydrogen, deuterium, fluorine, chlorine, bromine, iodine, methyl, ethyl, propyl, isopropyl, butyl, tert-butyl, and substituted C1-C6 alkyl, wherein the substituted C1-C6 alkyl is optionally further substituted with 1 to 6 substituents selected from deuterium, fluorine, chlorine, bromine, iodine, hydroxyl, carboxyl, amino, cyano, methyl, ethyl, propyl, and isopropyl.

In certain embodiments, R7 and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, and isopropyl.

Preferably, R2 is selected from hydrogen, deuterium, fluorine, chlorine, bromine, iodine, methyl, ethyl, propyl, isopropyl, butyl, and tert-butyl.

Preferably, R3 and R3′ are each independently selected from hydrogen, deuterium, fluorine, chlorine, bromine, iodine, methyl, ethyl, propyl, isopropyl, butyl, and tert-butyl.

Preferably, R4 and R6 are each independently selected from hydrogen, deuterium, fluorine, chlorine, bromine, iodine, methyl, ethyl, propyl, isopropyl, butyl, and tert-butyl.

Preferably, R7 and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, and cyclohexyl.

More preferably, R2 is selected from hydrogen, deuterium, methyl, ethyl, propyl, isopropyl, butyl, and tert-butyl.

More preferably, R3 and R3′ are each independently selected from hydrogen, deuterium, methyl, ethyl, propyl, isopropyl, butyl, and tert-butyl.

More preferably, R4 and R6 are each independently selected from hydrogen, deuterium, methyl, ethyl, propyl, isopropyl, butyl, and tert-butyl.

More preferably, R7 and R8 are each independently selected from hydrogen, deuterium, methyl, ethyl, propyl, isopropyl, butyl, tert-butyl, cyclopropyl, cyclobutyl, cyclopentyl, and cyclohexyl.

Most preferably, R2 is selected from hydrogen and methyl.

Most preferably, R3 and R3′ are each independently selected from hydrogen and methyl.

Most preferably, R4 is selected from hydrogen, methyl, and ethyl; and R6 is selected from hydrogen, deuterium, methyl, and ethyl.

Most preferably, R7 and R8 are each independently selected from hydrogen, deuterium, methyl, ethyl, isopropyl, cyclopropyl, and cyclobutyl.

Even more preferably, in certain specific embodiments, the compound is of formula (III) or (IV), or a stereoisomer or a pharmaceutically acceptable salt thereof.

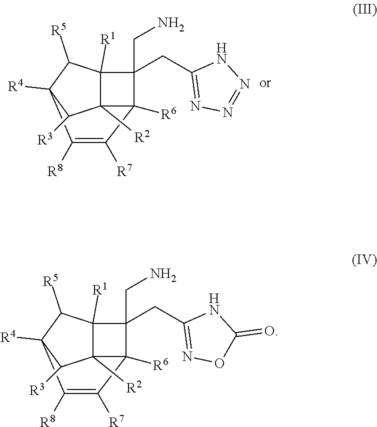

R1 and R5 are each independently selected from hydrogen, deuterium, methyl, ethyl, propyl, isopropyl, butyl, tert-butyl, and substituted C1-C6 alkyl, wherein the substituted C1-C6 alkyl is optionally further substituted with 1 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

R4, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

In certain embodiments, R2 is selected from hydrogen, deuterium, fluorine, chlorine, bromine, iodine, methyl, ethyl, propyl, isopropyl, butyl, tert-butyl, and substituted C1-C6 alkyl, wherein the substituted C1-C6 alkyl is optionally further substituted with 1 to 6 substituents selected from deuterium, fluorine, chlorine, bromine, iodine, hydroxyl, carboxyl, amino, cyano, methyl, ethyl, propyl, and isopropyl.

R3 and R3′ are each independently selected from hydrogen, deuterium, fluorine, chlorine, bromine, iodine, methyl, ethyl, propyl, isopropyl, butyl, tert-butyl, and substituted C1-C6 alkyl, wherein the substituted C1-C6 alkyl is optionally further substituted with 1 to 6 substituents selected from deuterium, fluorine, chlorine, bromine, iodine, hydroxyl, carboxyl, amino, cyano, methyl, ethyl, propyl, and isopropyl.

R4 and R6 are each independently selected from hydrogen, deuterium, fluorine, chlorine, bromine, iodine, methyl, ethyl, propyl, isopropyl, butyl, tert-butyl, and substituted C1-C6 alkyl, wherein the substituted C1-C6 alkyl is optionally further substituted with 1 to 6 substituents selected from deuterium, fluorine, chlorine, bromine, iodine, hydroxyl, carboxyl, amino, cyano, methyl, ethyl, propyl, and isopropyl.

R7 and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, and isopropyl.

Preferably, R2 is selected from hydrogen, deuterium, fluorine, chlorine, bromine, iodine, methyl, ethyl, propyl, isopropyl, butyl, and tert-butyl.

Preferably, R3 and R3′ are each independently selected from hydrogen, deuterium, fluorine, chlorine, bromine, iodine, methyl, ethyl, propyl, isopropyl, butyl, and tert-butyl.

Preferably, R4 and R6 are each independently selected from hydrogen, deuterium, fluorine, chlorine, bromine, iodine, methyl, ethyl, propyl, isopropyl, butyl, and tert-butyl.

Preferably, R7 and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, and cyclohexyl.

More preferably, R2 is selected from hydrogen, deuterium, methyl, ethyl, propyl, isopropyl, butyl, and tert-butyl.

More preferably, R3 and R3′ are each independently selected from hydrogen, deuterium, methyl, ethyl, propyl, isopropyl, butyl, and tert-butyl.

More preferably, R4 and R6 are each independently selected from hydrogen, deuterium, methyl, ethyl, propyl, isopropyl, butyl, and tert-butyl.

More preferably, R7 and R8 are each independently selected from hydrogen, deuterium, methyl, ethyl, propyl, isopropyl, butyl, tert-butyl, cyclopropyl, cyclobutyl, cyclopentyl, and cyclohexyl.

Most preferably, R2 is selected from hydrogen and methyl.

Most preferably, R3 and R3′ are each independently selected from hydrogen and methyl.

Most preferably, R4 is selected from hydrogen, methyl, and ethyl; and R6 is selected from hydrogen, deuterium, methyl, and ethyl.

Most preferably, R7 and R8 are each independently selected from hydrogen, deuterium, methyl, ethyl, isopropyl, cyclopropyl, and cyclobutyl.

The present invention provides a compound that is represented by any one of the following structures, or a stereoisomer or a pharmaceutically acceptable salt thereof:

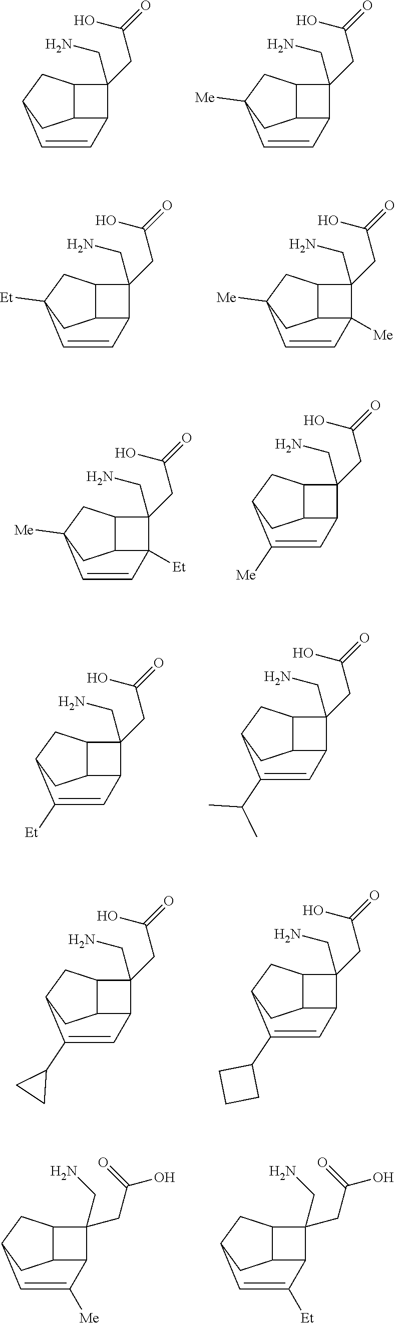

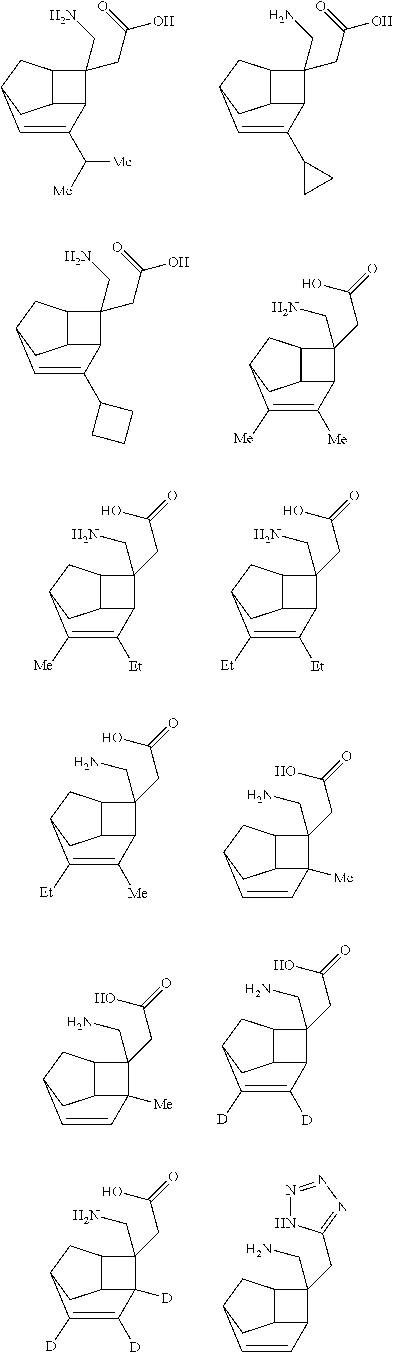

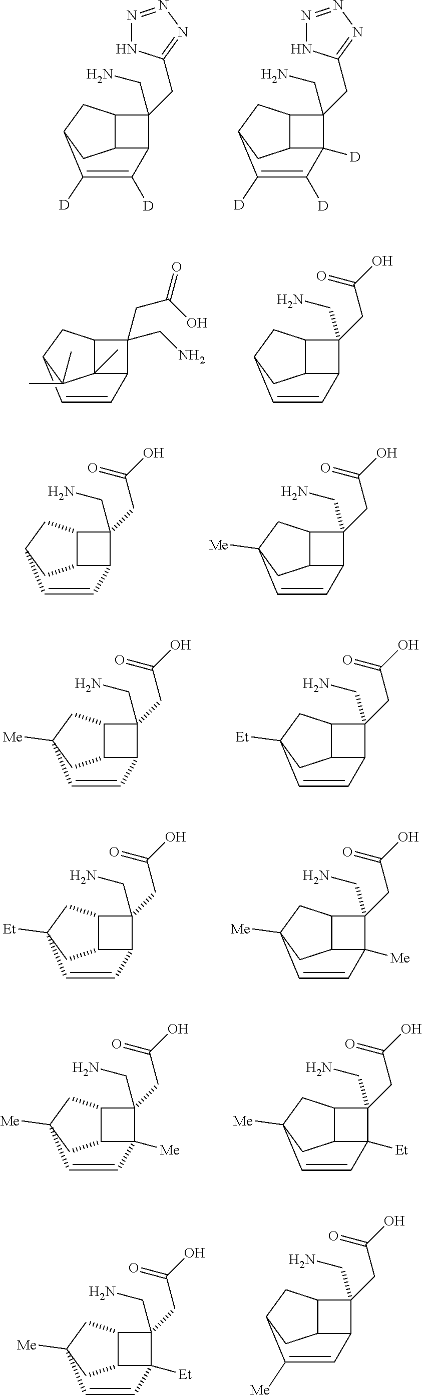

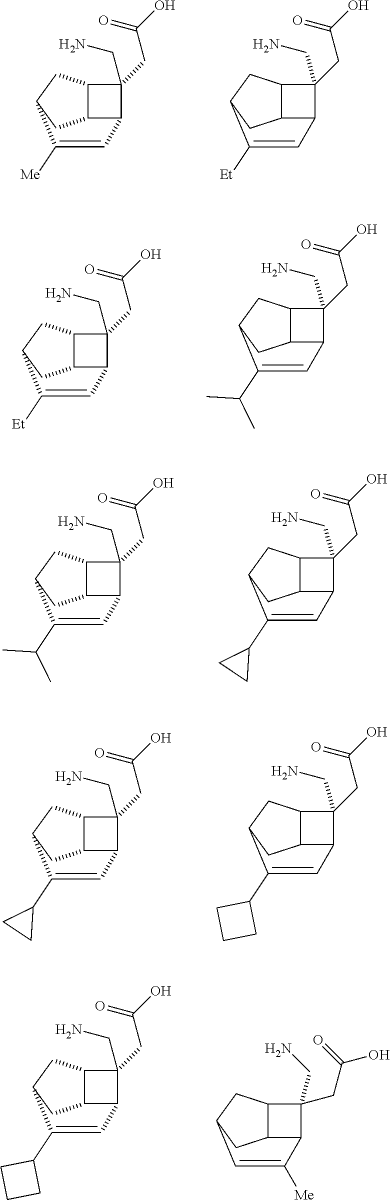

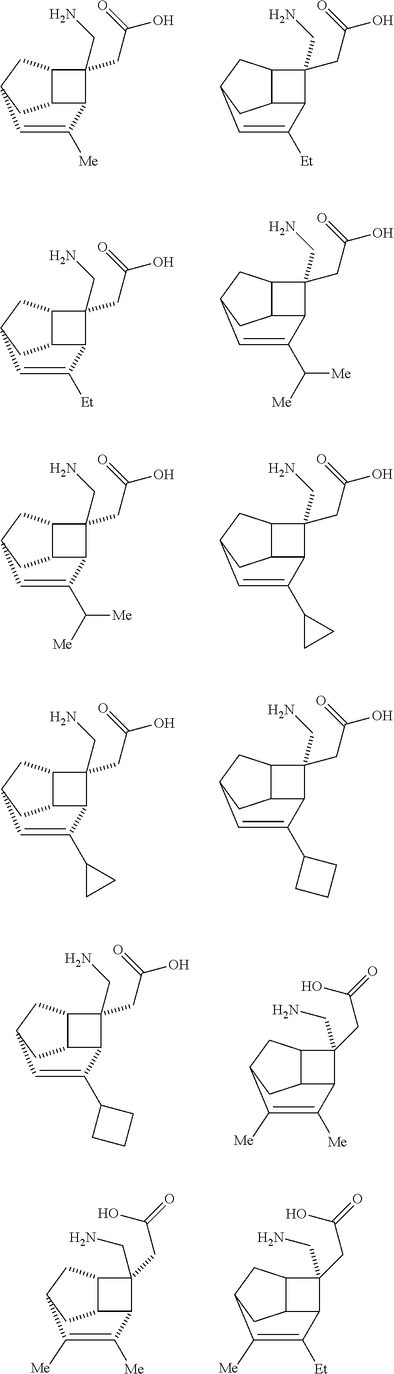

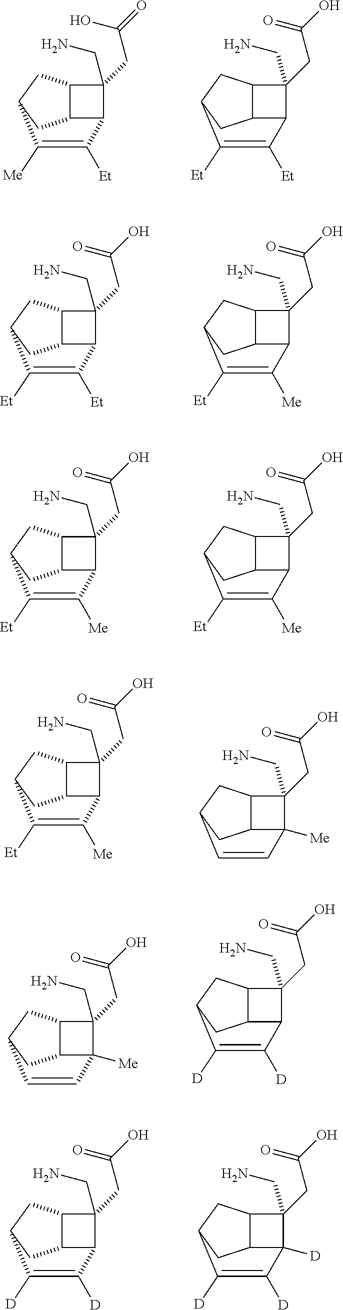

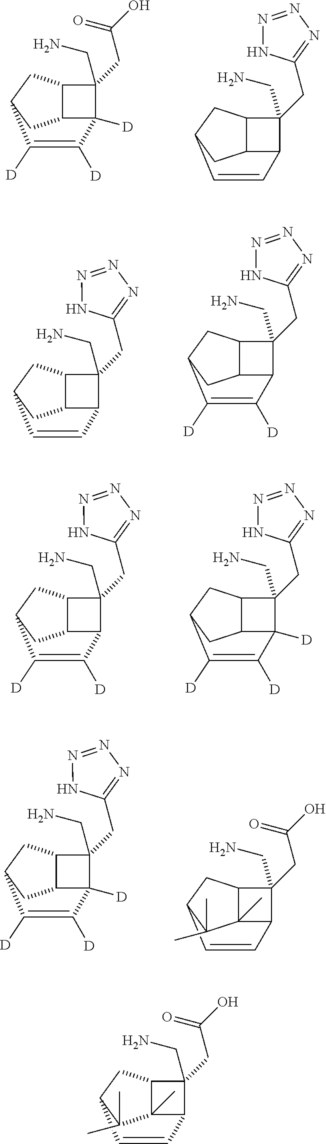

### Second Aspect:

The present invention further provides a pharmaceutical composition comprising a compound of formula (I) as described herein, a stereoisomer thereof, or a pharmaceutically acceptable salt thereof, or a mixture of the foregoing, together with at least one pharmaceutically acceptable excipient.

In certain embodiments, the compound of formula (I) is present in the pharmaceutical composition in a therapeutically effective amount.

### Third Aspect:

The present invention also provides the use of a compound of formula (I), a stereoisomer thereof, a pharmaceutically acceptable salt thereof, or the pharmaceutical composition described herein, for the preparation of a medicament for the prevention and/or treatment of diseases or disorders associated with ligands of the α2δ subunit of voltage-gated calcium channels.

The α2δ subunit-associated diseases or disorders are related to pain, including but not limited to: postherpetic neuralgia, trigeminal neuralgia, migraine, pain associated with osteoarthritis or rheumatoid arthritis, low back pain, sciatica, toothache, burn-induced pain, diabetic neuropathy, chemotherapy-induced neuropathic pain, HIV-related neuropathic pain, AIDS-related neuropathic pain, cancer-related neuropathic or non-neuropathic pain, acute or chronic tension headache, postoperative pain, fibromyalgia, epilepsy, generalized anxiety disorder, or restless legs syndrome.

Preferably, the pain is peripheral neuropathic pain, preferably diabetic peripheral neuropathy and postherpetic neuralgia.

### Fourth Aspect:

The present invention further provides the use of a compound of formula (I), a stereoisomer thereof, a pharmaceutically acceptable salt thereof, or the pharmaceutical composition described herein, for the preparation of a ligand targeting the α2δ subunit of voltage-gated calcium channels.

### Fifth Aspect:

The present invention also provides the use of a compound of formula (I), a stereoisomer thereof, a pharmaceutically acceptable salt thereof, or the pharmaceutical composition described herein, for the preparation of a medicament for the prevention and/or treatment of pain.

The pain may be selected from: postherpetic neuralgia, trigeminal neuralgia, migraine, pain associated with osteoarthritis or rheumatoid arthritis, low back pain, sciatica, toothache, burn-induced pain, diabetic neuropathy, chemotherapy-induced neuropathic pain, HIV-related neuropathic pain, AIDS-related neuropathic pain, cancer-related neuropathic or non-neuropathic pain, acute or chronic tension headache, postoperative pain, fibromyalgia, epilepsy, generalized anxiety disorder, or restless legs syndrome.

Preferably, the pain is peripheral neuropathic pain, and more preferably diabetic peripheral neuropathy and postherpetic neuralgia.

The present invention relates to an intermediate for the preparation of a compound of general formula (V), or a stereoisomer or a pharmaceutically acceptable salt thereof, wherein:

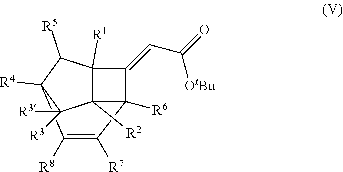

R1, R2, R3, R3′, R4, R5, R6, R7, and R8 are each independently selected from hydrogen, deuterium, halogen, substituted or unsubstituted C1-C6 alkyl, and substituted or unsubstituted C3-C6 cycloalkyl, wherein the substituted C1-C6 alkyl and substituted C3-C6 cycloalkyl are optionally further substituted with 1 to 6 substituents selected from halogen, hydroxyl, carboxyl, amino, cyano, C1-C3 alkyl, and C3-C6 cycloalkyl.

In certain specific embodiments, the compound is of formula (V-1), or a stereoisomer or a pharmaceutically acceptable salt thereof:

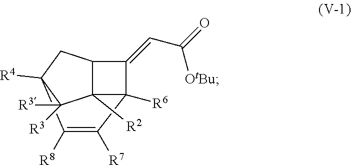

R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl; and
R4, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

In certain embodiments, R2 is selected from hydrogen, deuterium, fluorine, chlorine, bromine, iodine, methyl, ethyl, propyl, isopropyl, butyl, tert-butyl, and substituted C1-C6 alkyl, wherein the substituted C1-C6 alkyl is optionally further substituted with 1 to 6 substituents selected from deuterium, fluorine, chlorine, bromine, iodine, hydroxyl, carboxyl, amino, cyano, methyl, ethyl, propyl, and isopropyl.

R3 and R3′ are each independently selected from hydrogen, deuterium, fluorine, chlorine, bromine, iodine, methyl, ethyl, propyl, isopropyl, butyl, tert-butyl, and substituted C1-C6 alkyl, wherein the substituted C1-C6 alkyl is optionally further substituted with 1 to 6 substituents selected from deuterium, fluorine, chlorine, bromine, iodine, hydroxyl, carboxyl, amino, cyano, methyl, ethyl, propyl, and isopropyl.

R4 and R6 are each independently selected from hydrogen, deuterium, fluorine, chlorine, bromine, iodine, methyl, ethyl, propyl, isopropyl, butyl, tert-butyl, and substituted C1-C6 alkyl, wherein the substituted C1-C6 alkyl is optionally further substituted with 1 to 6 substituents selected from deuterium, fluorine, chlorine, bromine, iodine, hydroxyl, carboxyl, amino, cyano, methyl, ethyl, propyl, and isopropyl.

R7 and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 1 to 6 substituents selected from deuterium, fluorine, chlorine, bromine, iodine, hydroxyl, carboxyl, amino, cyano, methyl, ethyl, propyl, and isopropyl.

Preferably, R2 is selected from hydrogen, deuterium, fluorine, chlorine, bromine, iodine, methyl, ethyl, propyl, isopropyl, butyl, and tert-butyl.

Preferably, R3 and R3′ are each independently selected from hydrogen, deuterium, fluorine, chlorine, bromine, iodine, methyl, ethyl, propyl, isopropyl, butyl, and tert-butyl.

Preferably, R4 and R6 are each independently selected from hydrogen, deuterium, fluorine, chlorine, bromine, iodine, methyl, ethyl, propyl, isopropyl, butyl, and tert-butyl.

Preferably, R7 and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, and cyclohexyl.

More preferably, R2 is selected from hydrogen, deuterium, methyl, ethyl, propyl, isopropyl, butyl, and tert-butyl.

More preferably, R3 and R3′ are each independently selected from hydrogen, deuterium, methyl, ethyl, propyl, isopropyl, butyl, and tert-butyl.

More preferably, R4 and R6 are each independently selected from hydrogen, deuterium, methyl, ethyl, propyl, isopropyl, butyl, and tert-butyl.

More preferably, R7 and R8 are each independently selected from hydrogen, deuterium, methyl, ethyl, propyl, isopropyl, butyl, tert-butyl, cyclopropyl, cyclobutyl, cyclopentyl, and cyclohexyl.

Most preferably, R2 is selected from hydrogen and methyl.

Most preferably, R3 and R3′ are each independently selected from hydrogen and methyl.

Most preferably, R4 is selected from hydrogen, methyl, and ethyl; and R6 is selected from hydrogen, deuterium, methyl, and ethyl.

Most preferably, R7 and R8 are each independently selected from hydrogen, deuterium, methyl, ethyl, isopropyl, cyclopropyl, and cyclobutyl.

In a preferred embodiment, the present invention provides a compound of general formula (V), or a stereoisomer or a pharmaceutically acceptable salt thereof, wherein the compound is selected from one of the following structures:

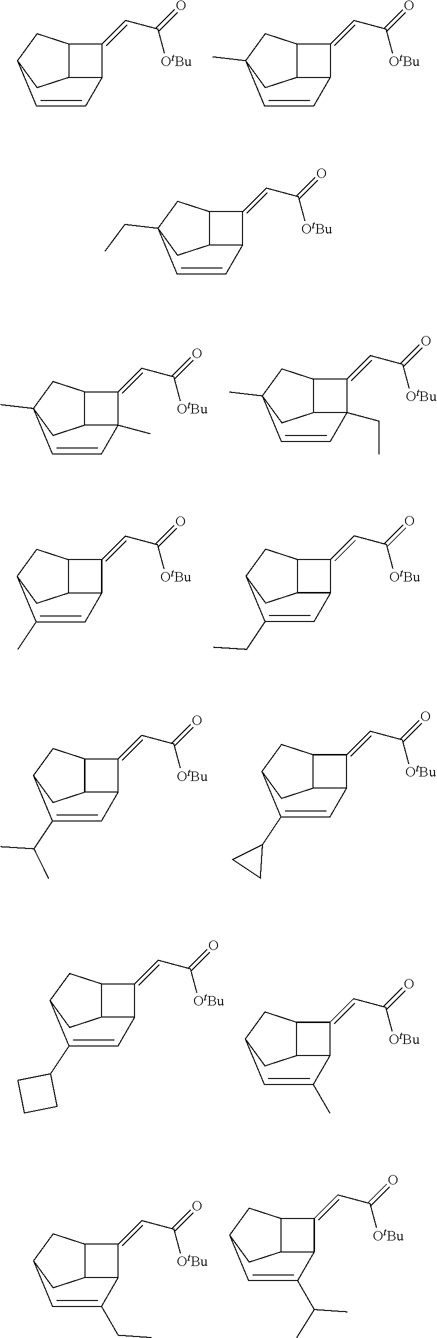

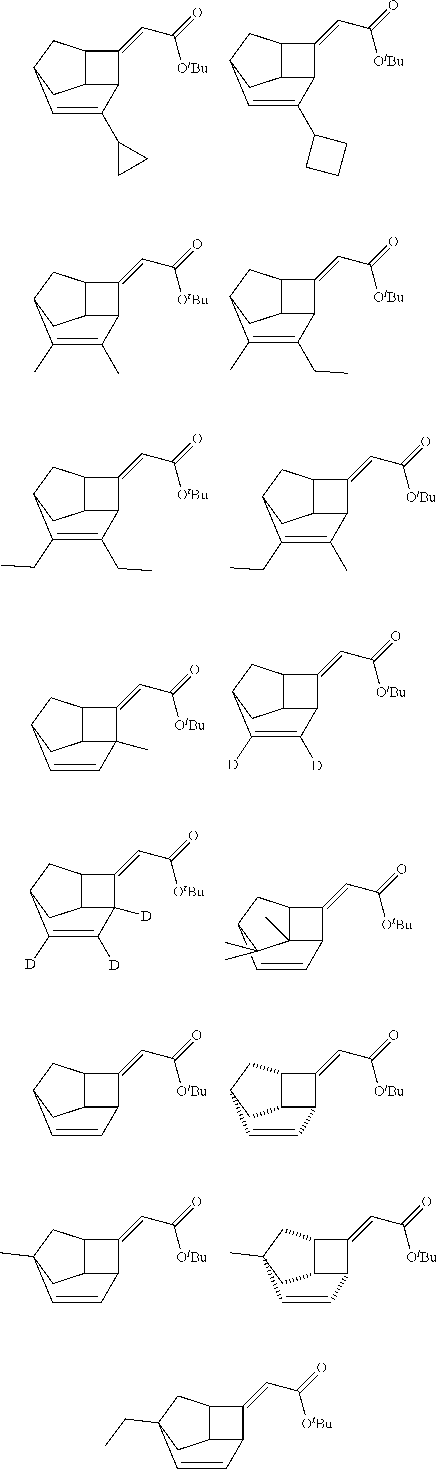

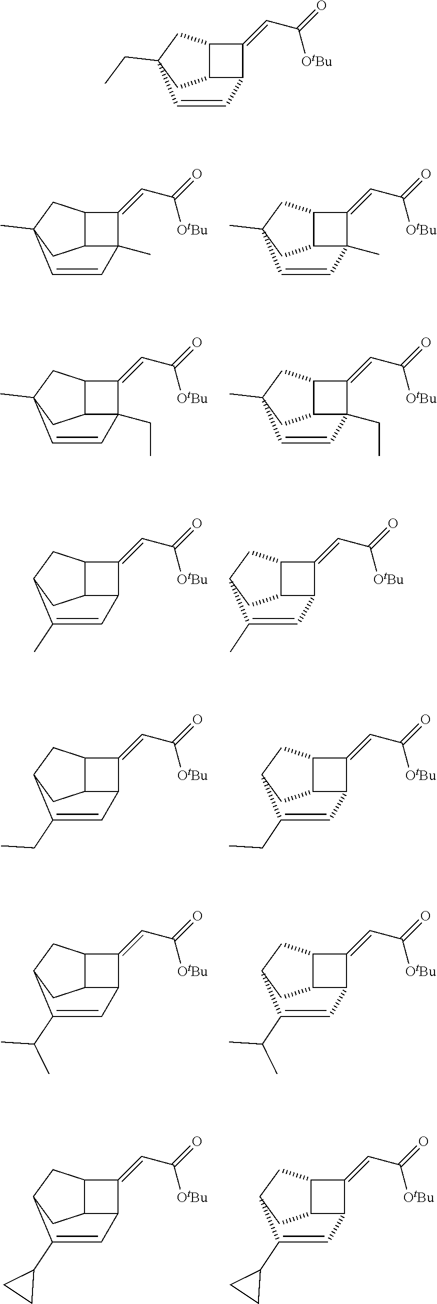

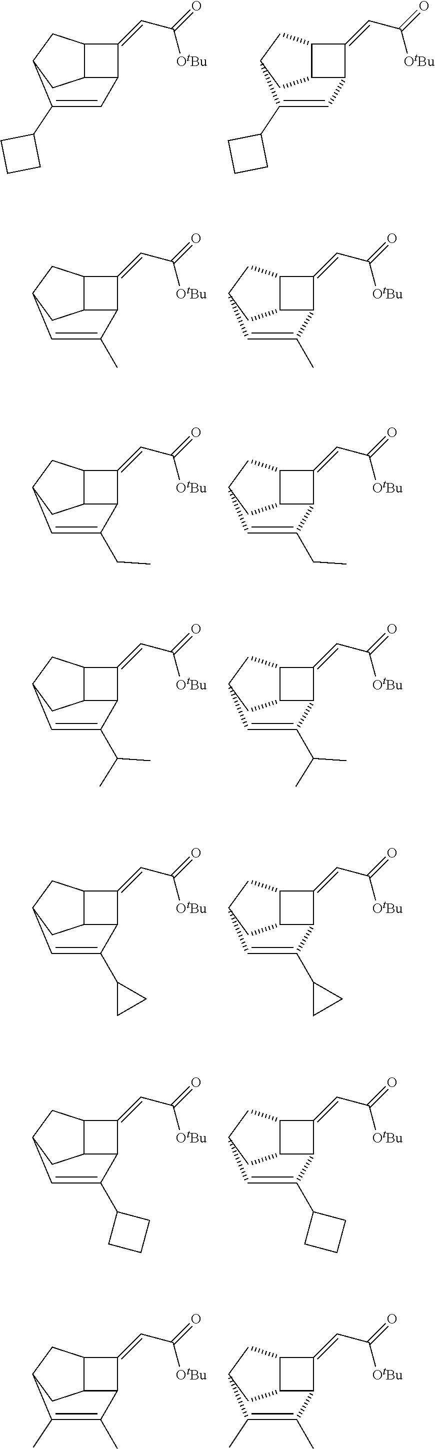

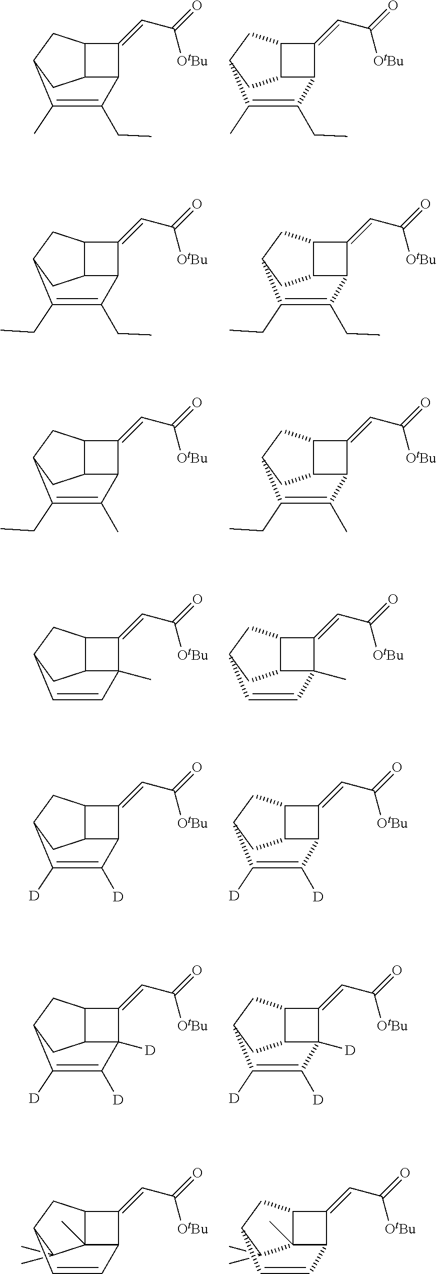

Unless otherwise specified, the definitions of groups and terms set forth in this specification and the claims—including those provided by way of example, exemplification, preference, tabular listing, or in reference to specific compounds in the embodiments—may be combined or interchanged in any manner. Accordingly, subsequent definitions of groups and compound structures are to be understood as being within the scope of the disclosure.

The compounds described herein may possess one or more asymmetric centers. Compounds of the invention that contain asymmetric substituents may exist in optically active or racemic forms. Unless a specific stereochemistry or isomeric form is explicitly indicated, all chiral, non-enantiomeric, racemic, and geometrical isomeric forms of the structures are intended to be encompassed by the present disclosure.

As used herein, numerical ranges defined for substituents—such as 1-6, 1-3, and 3-6—refer to integers within the stated range, inclusive. For example, 1-6 means 1, 2, 3, 4, 5, or 6.

The term “halogen” refers to fluorine, chlorine, bromine, or iodine, and is preferably fluorine.

The term “alkyl” refers to a monovalent saturated hydrocarbon group, either straight-chain or branched. For example, “C1-C6 alkyl” refers to a saturated hydrocarbon group having 1 to 6 carbon atoms, which may be linear or branched. Similarly, “C1-C3 alkyl” refers to such a group having 1 to 3 carbon atoms.

Examples of alkyl groups include, but are not limited to: methyl, ethyl, n-propyl, isopropyl, n-butyl, tert-butyl, sec-butyl, isobutyl, n-pentyl, 3-pentyl, pentyl, neopentyl, 3-methyl-2-butyl, tert-pentyl, and n-hexyl.

In the present application, the alkyl group is preferably a C1-C3 alkyl.

The term “haloalkyl” refers to an alkyl group as defined above in which one or more hydrogen atoms are replaced by halogen atoms. The number of halogen substituents may be 1, 2, 3, 4, or 5.

In the present application, the haloalkyl group is preferably a C1-C3 haloalkyl.

Specific examples of haloalkyl groups include but are not limited to: —CH₂F, —CH₂Cl, —CHF₂, —CHCl₂, —CCl₃, —CF₃, —CH₂CH₂F, —CH₂CHF₂, —CH₂CF₃, and —CF₂CF₃.

The term “cycloalkyl” refers to a saturated carbocyclic hydrocarbon group composed of the specified number of ring carbon atoms (e.g., C3-C6, C3-C8). For example, “C3-C6 cycloalkyl” refers to a saturated cyclic hydrocarbon group containing 3 to 6 ring carbon atoms. Exemplary cycloalkyl groups include, but are not limited to: cyclopropyl, cyclobutyl, cyclopentyl, and cyclohexyl.

The term “stereoisomer” as used herein refers to isomers that differ in the spatial arrangement of atoms within the molecule, and includes cis-trans isomers, enantiomers, and conformational isomers.

The term “pharmaceutically acceptable salt” refers to an organic or inorganic salt of the compound of the present invention, as defined above, which retains the desired pharmacological activity. Such salts include acid addition salts formed with inorganic or organic acids. Pharmaceutically acceptable salts also include base addition salts, which may be formed in the presence of an acidic proton capable of reacting with an inorganic or organic base.

As used herein, when any variable (e.g., R) appears more than once in a compound's composition or structure, each occurrence is defined independently.

For example, if a group is said to be substituted with 1-2 R groups, the group may optionally be substituted with up to two R groups, and each R may be selected independently.

Furthermore, combinations of substituents and/or their variants are only permitted to the extent that such combinations result in stable compounds.

The term “therapeutically effective amount” as used herein refers to an amount of a compound that is sufficient to effectively treat a disease in a subject.

The therapeutically effective amount may vary depending on factors such as the specific compound, the type and severity of the disease, the age of the patient, and other clinical parameters, and can be determined and adjusted as needed by those skilled in the art.

The term “pharmaceutically acceptable excipient” as used herein refers to any substance included in a pharmaceutical formulation other than the active pharmaceutical ingredient (API). Such excipients are generally classified into two categories: formulation excipients and additives. For further details, reference may be made to the Pharmacopoeia of the People's Republic of China (2020 Edition) [Handbook of Pharmaceutical Excipients (Paul J Sheskey, Bruno C Hancock, Gary P Moss, David J Goldfarb, 2020, 9th Edition).

The term “treatment” as used herein refers to the elimination of the cause of a disease or the alleviation of its symptoms.

The term “prevention” refers to the reduction in the risk of occurrence of a disease.

The term “patient” or “subject” refers to any animal in need of treatment or prevention of a disease, typically a mammal, such as a human. Mammals include, but are not limited to cattle, horses, sheep, pigs, cats, dogs, mice, rats, rabbits, guinea pigs, monkeys, and humans.

Other features and advantages of the present invention will be set forth in the following description. In part, it will be apparent from the description or may be learned by practice of the invention. The objectives & advantages of the invention will be realized and attained by means of the structures particularly pointed out in the description, the claims, and the accompanying drawings.

The above-mentioned preferred conditions may be combined in any manner, so long as such combinations are consistent with the common general knowledge in the field, thereby yielding various preferred embodiments of the present invention.

All the reagents and starting materials used in the present invention are commercially available.

The advantageous effects of the present invention are as follows: the compounds of the present invention possess novel chemical structures, and in vitro assays have demonstrated their strong affinity for the α2δ subunit of voltage-gated calcium channels. Furthermore, the compounds exhibit favorable analgesic activity in vivo and are therefore suitable for the preparation of medicaments for treating and/or alleviating diseases or conditions associated with α2δ subunit ligands of voltage-gated calcium channels.

Specifically, the diseases or conditions are associated with pain, which includes, but is not limited to: postherpetic neuralgia, trigeminal neuralgia, migraine, pain associated with osteoarthritis or rheumatoid arthritis, low back pain, sciatica, toothache, burn-induced pain, pain caused by diabetic neuropathy, diabetic peripheral neuropathic pain, diabetic peripheral neuropathy, diabetic peripheral neuralgia, chemotherapy-induced neuropathic pain, neurological disorders, central neuropathic pain, central nervous system-related pain, peripheral nervous system disorders, peripheral neuralgia, HIV-related neuralgia, AIDS-related neuralgia, cancer-related neuropathic or non-neuropathic pain, acute or chronic tension-type headache, postoperative pain, fibromyalgia, epilepsy, generalized anxiety disorder, restless legs syndrome, chronic kidney disease, renal insufficiency, and perioperative orthopedic analgesia.

Preferably, the pain is peripheral neuropathic pain, and most preferably diabetic peripheral neuropathy and postherpetic neuralgia.

### EXAMPLES

### Synthetic Example

Instrumentation and Analytical Methods: ¹H NMR spectra were recorded on a BrukerAvance Neo 400 MHz superconducting NMR spectrometer operating at 400 MHz. Chemical shifts (5) are reported in parts per million (ppm) using tetramethylsilane (TMS, δ=0) as the internal standard. CDCl₃, MeOD-d₄, and D₂O were used as solvents.

Mass spectra were collected using a Waters ACQUITY UPLC system equipped with an ACQUITY UPLC BEH C8 column (50 mm×2.1 mm, 1.7 μm, 20180306-C8-08). Mobile phase A: 0.01% TFA in H₂O; Mobile phase B: acetonitrile (CH₃CN); Flow rate: 0.2 mL/min; Column temperature: 30° C.; Detection wavelength: UV at 210 nm.

High-performance liquid chromatography (HPLC) analysis was performed on a Thermo UltiMate 3000 system using a Venusil ASB C18 column (4.6×250 mm, 5 μm). Mobile phase A: phosphoric acid aqueous solution (pH=1.5); Mobile phase B: acetonitrile (CH₃CN); Flow rate: 1.0 mL/min; Column temperature: 35° C.; Detection wavelength: UV at 215 nm; Injection volume: 2 μL.

Gradient elution conditions: −0-10 min: 95% A/5% B; −10-15 min: 20% A/80% B; −15-20 min: 95% A/5% B.

All percentage values refer to the volume ratio of the mobile phases in the eluent.

### Example 1: Preparation of Key Intermediate 1-1

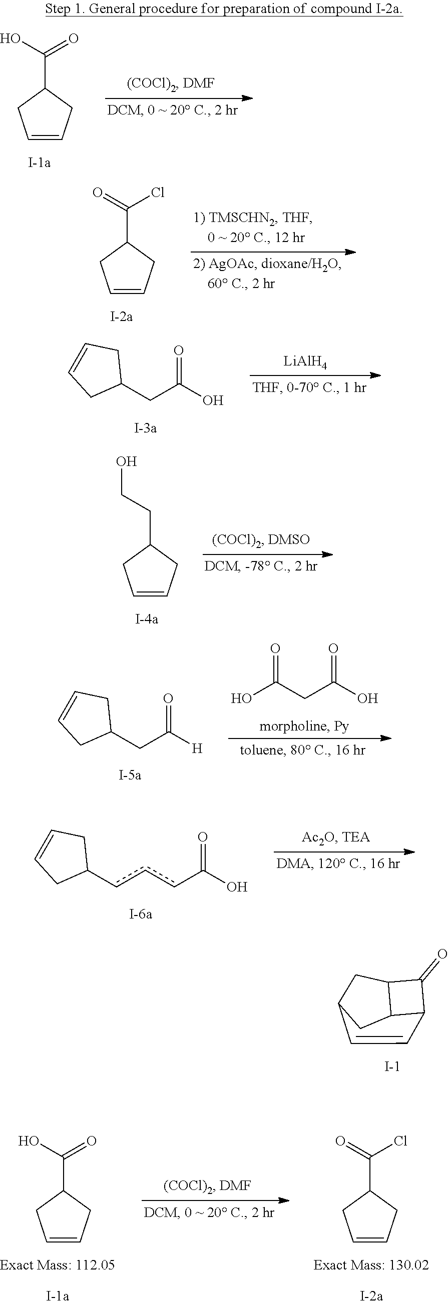

To a solution of I-1a (40.0 g, 356 mmol) and DMF (2.61 g, 35.67 mmol, 2.74 mL) in DCM (1200 mL) was added (COCl)₂ (90.5 g, 713 mmol, 62.4 mL) dropwise at 0° C., the mixture was stirred at 20° C. for 2 hours. After the reaction was completed, the mixture was concentrated under reduced pressure to give compound I-2a (40 g, 306.34 mmol, crude) as a brown oil, which was used to the next step directly without further purification.

### Step 2. General Procedure for Preparation of Compound I-3a.

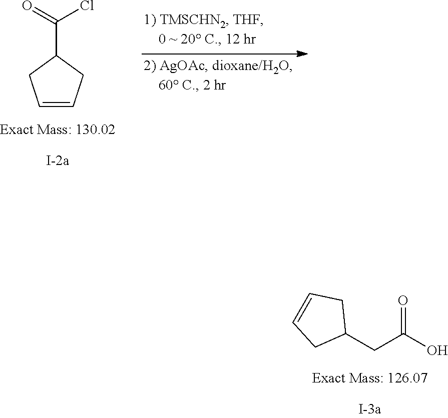 [SMILES: O=C(Cl)C1CC=CC1 ; O=C(O)CC1CC=CC1]

To a solution of I-2a (12.0 g, 75.6 mmol) (3 batches) in THF (1500 mL) was added TMSCHN₂ (2 M in hexanes, 382.93 mL) dropwise at 0° C., the mixture was stirred at 20° C. for 12 hours. TLC (Petroleum ether:Ethyl acetate=5:1 new spot Rf=0.8) showed compound I-3a was consumed completely and one new spot was formed. The reaction mixture was concentrated under reduced pressure to remove the solvent to give a residue, then the residue was dissolved with 1,4-dioxane (800 mL) and H₂O (800 mL), AgOAc (51.13 g, 306.34 mmol, 15.69 mL, 1 eq) was added, the mixture was stirred at 60° C. for another 2 hours. After the reaction was completed, the solution was adjusted to pH=13 by 1 M NaOH, the mixture was washed with EtOAc (500 mL×3). Then the inorganic phase was adjusted to pH=3 by 1 M HCl, extracted with EtOAc (500 mL×3), the organic phase was washed with brine (500 mL), dried over Na₂SO₄ and concentrated under reduced pressure. The residue was purified by column chromatography (SiO₂, Petroleum ether:Ethyl acetate=10:0 to 5:1) to give I-3a (37.0 g, 293 mmol, 95.7% yield) as a colorless oil.

¹H NMR: (400 MHz, CDCl₃)

δ=5.73-5.62 (m, 2H), 2.74-2.65 (m, 2H), 2.65-2.55 (m, 2H), 2.46 (s, 2H), 2.09-2.05 (m, 1H)

### Step 3. General Procedure for Preparation of Compound I-4a.

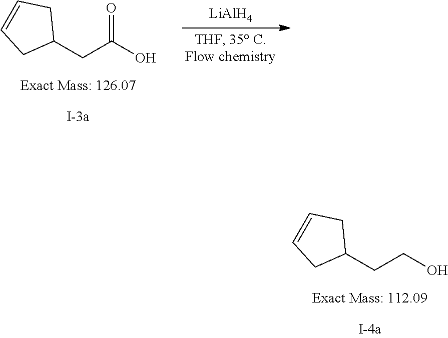 [SMILES: O=C(O)CC1CC=CC1 ; "LiAlH4" ; OCCC1CC=CC1]

Solution 1: I-3a (35 g, 277 mmol) was filled with THF (650 mL).

Solution 2: LiAlH₄ (2.5 M solution in THF, 166.46 mL).

The solution 1 was pumped by Pump 1 S1, P1, 8.864 mL/min to flow reactor 1 FLR1, PFA, Coils reactor, 3.175(⅛″) mm, 68.06 mL, 35° C. under N₂ atmosphere.

The solution 2 was pumped by Pump 2 S2, P2, 2.162 mL/min to flow reactor 1 FLR1, PFA, Coils reactor, 3.175(⅛″) mm, 68.06 mL, 35° C. under N₂ atmosphere.

The residence time of flow reactor 1 was FLR1, 10 min. The mixture was collected with a bottle. The Pump1 and Pump2 were started at the same time. The reaction mixture was collected after running 10 mins. Stop collecting the reaction mixture after 60 mins. The mixture was quenched with Na₂SO₄·10H₂O (50 g) at 0° C. under N₂ and stirred at 25° C. for 10 minutes. Then the mixture was filtered through a pad of celite and the solution was concentrated under reduced pressure give compound I-4a (30.0 g, 267 mmol, 96.4% yield) as a colorless oil.

¹H NMR: (400 MHz, CDCl₃)

δ=5.69 (s, 2H), 3.73-3.66 (m, 2H), 2.56-2.46 (m, 2H), 2.41-2.30 (m, 1H), 2.10-1.95 (m, 2H), 1.77-1.66 (m, 2H).

### Step 4. General Procedure for Preparation of Compound I-5a.

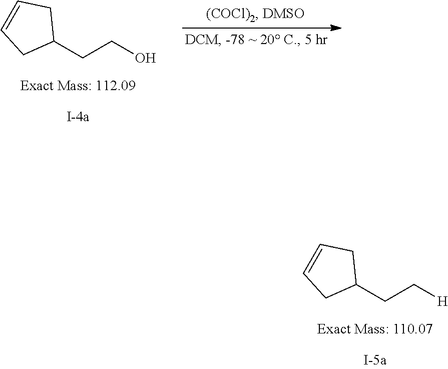

To a solution of (COCl)₂ (47.5 g, 374 mmol, 32.7 mL) in DCM (450 mL) was added DMSO (58.5 g, 748 mmol, 58.5 mL) in DCM (150 mL) dropwise at −78° C. for 2 hours under N₂, then I-4a (30.0 g, 267 mmol) in DCM (450 mL) was added dropwise at −78° C. under N₂ for 2 hours, then TEA (75.7 g, 748 mmol, 104.23 mL) was added at −78° C. under N₂, the mixture was stirred at 20° C. for 1 hour. After the reaction was completed, the mixture was diluted with water (500 mL), extracted with MTBE (200 mL×3), the organic phase was dried over Na₂SO₄ and concentrated under reduced pressure at 20° C. to give compound I-5a (30 g, crude) as a yellow solid.

### Step 5. General Procedure for Preparation of Compound I-6a.

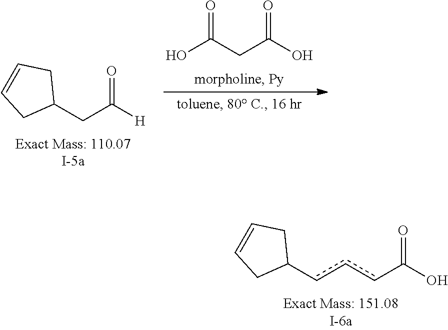

To a solution of I-5a (30.0 g, 272 mmol) in toluene (300 mL) was added morpholine (14.2 g, 163 mmol, 14.3 mL), malonic acid (42.5 g, 408 mmol, 42.5 mL) and pyridine (49.5 g, 626 mmol, 50.5 mL), the mixture was stirred at 80° C. for 16 hours. After the reaction was completed, the mixture was diluted with water (200 mL), then the solution was adjust to pH=13 by 1 M NaOH. The mixture was washed with EtOAc (200 mL×3), the inorganic phase was adjust to pH=3 by 1 M HCl, Then the solution was extracted EtOAc (200 mL×3), washed with brine (500 mL), dried over Na₂SO₄ and concentrated under reduced pressure. The residue was purified by column chromatography (SiO₂, Petroleum ether:Ethyl acetate=10:1 to 5:1) to give compound I-6a (26.0 g, 170 mmol, 62.7% yield) as a yellow oil.

LC-MS: m/z=151.0, M−H⁺

¹H NMR: (400 MHz, CDCl₃)

δ=7.19-7.01 (m, 1H), 5.89-5.80 (m, 1H), 5.72-5.65 (m, 2H), 4.13 (q, J=7.2 Hz, 1H), 2.59-2.48 (m, 2H), 2.47-2.38 (m, 1H), 2.34-2.30 (m, 1H), 2.07-1.99 (m, 3H), 1.27 (t, J=7.2 Hz, 1H)

### Step 6. General Procedure for Preparation of Compound I-1.

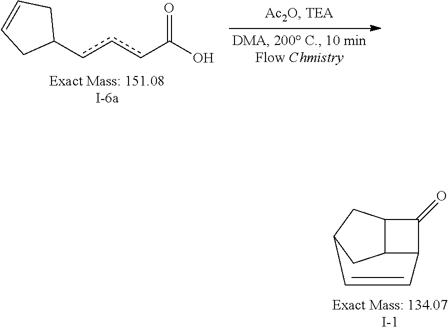

A solution of I-6a (23.4 g, 153 mmol), Ac₂O (28.2 g, 276 mmol, 25.9 mL) and TEA (17.1 g, 169 mmol, 23.5 mL) in DMA (1200 mL) was running 12 mins under 200° C. by flow chemistry. After the reaction was completed, the mixture was diluted with water (2000 mL), extracted with EtOAc (1000 mL×3), the organic phase was washed with brine (500 mL×3), dried over Na₂SO₄ and concentrated under reduced pressure. The residue was purified by column chromatography (SiO₂, Petroleum ether:Ethyl acetate=20:1 to 10:1) to give compound 1-1 (12.0 g, 71.5 mmol, 46.5% yield, 80% purity) as a brown oil.

LC-MS: m/z=135.3, M+H⁺

¹H NMR: (400 MHz, CDCl₃)

δ=6.46 (t, J=8.4 Hz, 1H), 5.65 (dd, J=6.8, 8.8 Hz, 1H), 3.64 (q, J=6.8 Hz, 1H), 3.42 (td, J=6.4, 10.8 Hz, 1H), 2.99 (q, J=5.4 Hz, 1H), 2.87-2.76 (m, 1H), 2.28 (d, J=12.8 Hz, 1H), 2.08 (ddd, J=5.2, 10.4, 12.8 Hz, 1H), 1.79 (td, J=4.0, 11.2 Hz, 1H), 1.47 (d, J=11.2 Hz, 1H)

### Example 2: Preparation of Key Intermediate II-1

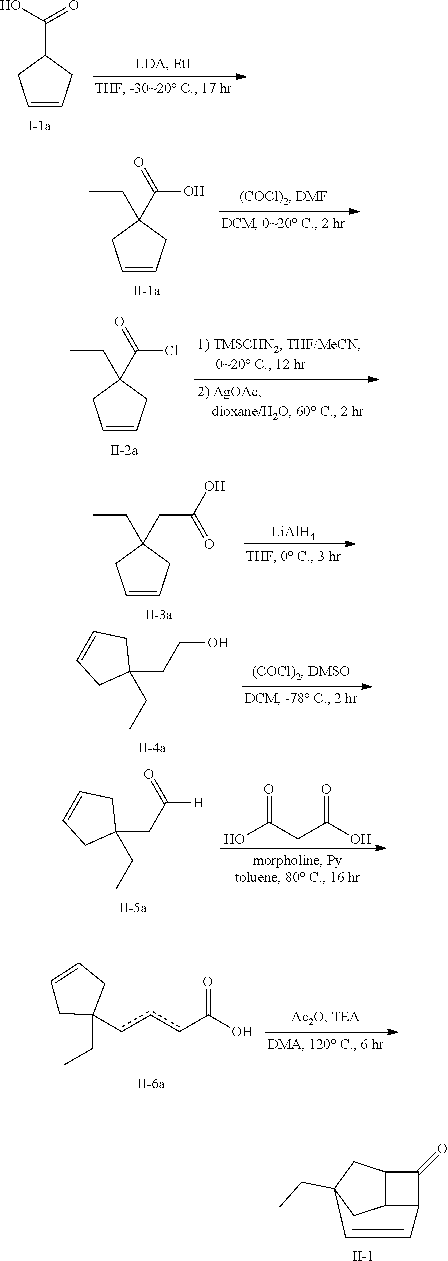

### Step 1. General Procedure for Preparation of Compound II-1a.

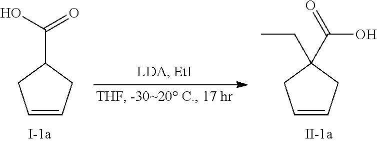

To a solution of LDA (2 M, 278 mL) in THF (500 mL) was added a solution of compound I-1a (25.0 g, 222 mmol) in THF (125 mL) dropwise at −30° C. under N₂, the mixture was warmed to 20° C. for 12 hours under N₂. Then the mixture was cooled to −30° C., Etl (34.7 g, 222 mmol) was added dropwise at −30° C. under N₂, the mixture was warmed to 20° C. for 2 hours under N₂. TLC (Petroleum ether:Ethyl acetate=5:1 new spot Rf=0.5) showed compound I-1a was consumed completely and one new spot was detected. After the reaction was completed, the mixture was quenched with 3M HCl (200 mL) to pH=5 under N₂, and extracted with EtOAc (1000 mL×3). The combined organic extracts were washed with brine (200 mL), dried over Na₂SO₄ and concentrated under reduced pressure. The residue was purified by column chromatography (SiO₂, Petroleum ether:Ethyl acetate=10:0 to 5:1) to give compound II-1a (28.0 g, 89% yield) was obtained as brown oil.

¹H NMR: (400 MHz, CDCl₃)

δ=2.34-10.60 (m, 1H), 5.62 (s, 2H), 2.98-2.86 (m, 2H), 2.33 (d, J=14.4 Hz, 2H), 1.76 (q, J=7.6 Hz, 2H), 0.91 (t, J=7.6 Hz, 4H)

### Step 2. General Procedure for Preparation of Compound II-2a.

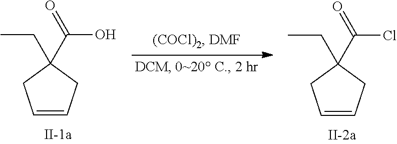

To a solution of compound II-1a (12.0 g, 85.6 mmol, two batches) and DMF (625 mg, 8.56 mmol) in DCM (300 mL) was added (COCl)₂ (21.7 g, 171 mmol) dropwise at 0° C., the mixture was stirred at 20° C. for 1 hour. TLC (Petroleum ether: Ethyl acetate=5:1 new spot Rf=0.6) showed compound II-1a was consumed completely and one new spot was detected. After the reaction was completed the reaction mixture was concentrated under reduced pressure to give compound II-2a (28 g, crude) was obtained as a brown oil.

### Step 3. General Procedure for Preparation of Compound II-3a.

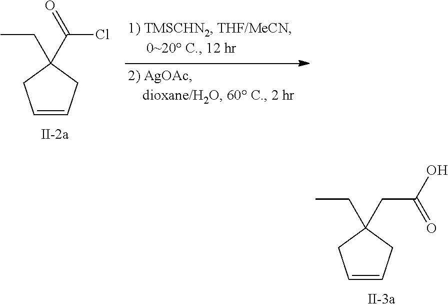

To a solution of compound II-2a (12.0 g, 75.6 mmol, two batches) in THF (500 mL) was added TMSCHN₂ (2 M, 117 mL) dropwise at 0° C., the mixture was stirred at 20° C. for 12 hours. TLC (Petroleum ether:Ethyl acetate=5:1 new spot Rf=0.8) showed compound W114-3a was consumed completely and one new spot was detected. The reaction mixture was concentrated under reduced pressure, then the residue was dissolved with Dioxane (150 mL) and H₂O (150 mL), AgOAc (5.30 g, 33.6 mmol), the mixture was stirred at 20° C. for another 2 hours. TLC (Petroleum ether:Ethyl acetate=5:1 new spot Rf=0.45) showed compound II-2a was consumed completely and one new spot was detected. After the reaction was completed, the mixture was quenched with 3M HCl (200 mL) to pH=5 under N₂, and extracted with EtOAc (1000 mL×3). The combined organic extracts were washed with brine (200 mL), dried over Na₂SO₄ and concentrated under reduced pressure. The residue was purified by column chromatography (SiO₂, Petroleum ether:Ethyl acetate=10:0 to 5:1) to give II-3a (6.5 g, 28% yield) was obtained as a colorless oil.

¹H NMR: (400 MHz, CDCl₃)

δ=11.15-9.78 (m, 1H), 5.69-5.52 (m, 2H), 2.47-2.42 (m, 2H), 2.41-2.20 (m, 4H), 1.62-1.54 (m, 2H), 0.92-0.87 (m, 3H)

### Step 4. General Procedure for Preparation of Compound II-4a.

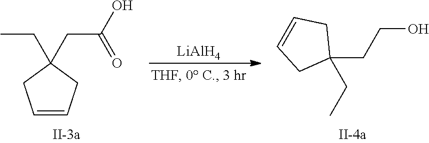

To a solution of compound II-3a (6.5 g, 42.15 mmol) in THF (180 mL) was added LiAlH₄ (2.5 M, 33.72 mL) dropwise at 0° C. under N₂, the mixture was stirred at 70° C. for 1 hr under N₂. TLC (Commercial hexanes:Ethyl acetate=5:1 new spot Rf=0.2) showed the reaction was completed. The mixture was quenched with Na₂SO₄·H₂O (20 g) and stirred at 25° C. for 10 minutes. Then the mixture was filtered through a pad of celite and the filtrate was concentrated under reduced pressure to give compound II-4a (5.1 g, 86% yield) as colorless oil, which was used to the next step without purification.

### Step 5. General Procedure for Preparation of Compound II-5a.

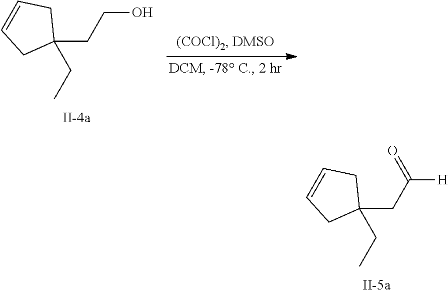

To a solution of (COCl)₂ (6.59 g, 51.92 mmol) in DCM (50 mL) was added DMSO (8.11 g, 103.84 mmol) in DCM (12 mL) dropwise at −78° C. for 30 min under N₂. then compound II-4a (5.2 g, 37.08 mmol) in DCM (50 mL) was added dropwise at −78° C. under N₂ for 30 min. Then TEA (10.51 g, 103.84 mmol) was added at −78° C. under N₂, the mixture was stirred at −78° C. for 0.5 h. TLC (Commercial hexanes:Ethyl acetate=5:1) showed the starting material was consumed, and one new spot was detected. The mixture was diluted with water (40 mL), extracted with EtOAc (50 mL*3). The combined organic extracts were dried over Na₂SO₄ and filtered, the filtrate was concentrated under reduced pressure at 20° C. to give compound II-5a (4.07 g, 79% yield) as colorless oil, which was used to the next step without purification.

### Step 6. General Procedure for Preparation of Compound II-6a.

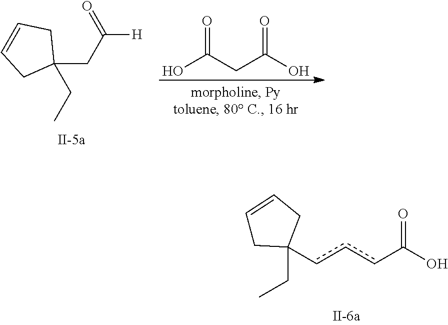

To a solution of malonic acid (4.60 g, 44.17 mmol) in toluene (40 mL) was added morpholine (1.54 g, 17.67 mmol), Pyridine (5.36 g, 67.73 mmol) and compound II-5a (4.07 g, 29.45 mmol) at 25° C. The reaction mixture was stirred for 16 h at 85° C. LCMS showed compound II-5a was consumed, the desired mass was detected. The pH of the reaction mixture was adjusted to 12 by using 2M NaOH and extracted with EtOAc (50 mL×2). The pH of water phase was adjusted to 3 by using 1 M HCl, the mixture was extracted with EtOAc (100 mL×2). The combined organic layers were washed with brine (100 mL), dried over Na₂SO₄, filtered and concentrated under reduced pressure. The residue was purified by a column on silica gel eluted with (Commercial hexanes:Ethyl acetate=10:0 to 5:1) to give compound II-6a (2.4 g, 45% yield) as yellow oil.

¹H NMR: (400 MHz, CDCl₃)

δ=7.06 (td, J=7.6, 15.2 Hz, 0.5H), 5.89-5.77 (m, 0.5H), 5.74-5.55 (m, 2H), 5.45 (td, J=6.8, 15.6 Hz, 1H), 3.10 (dd, J=1.2, 6.8 Hz, 1H), 2.38-2.18 (m, 4H), 1.60-1.41 (m, 2H), 0.84 (td, J=7.2, 17.6 Hz, 3H)

### Step 7. General Procedure for Preparation of Compound II-1.

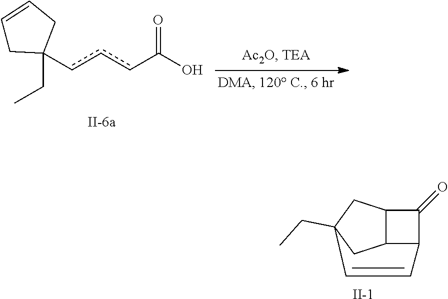

To a solution of compound II-6a (1.4 g, 7.77 mmol) in DMA (70 mL) was added Ac₂O (1.43 g, 13.98 mmol) and TEA (864 mg, 8.54 mmol) at 25° C. under N₂. The reaction mixture was stirred for 16 h at 120° C. TLC (PE:EtOAc=10:1) showed the starting material remained, and a new spot was formed. The mixture was diluted with H₂O (100 mL), the mixture was extracted with EtOAc (120 mL), the organic layer was washed with brine (60 mL×3), dried over Na₂SO₄, filtered and concentrated under reduced pressure (25° C.). The residue was purified by flash silica gel chromatography (12 g Silica Flash Column, Eluent of 0˜2% Ethylacetate/Commercial hexanes@30 mL/min) to give compound II-1 (600 mg, 47% yield) as yellow oil

¹H NMR: (400 MHz, CDCl₃)

δ=6.25 (d, J=9.2 Hz, 1H), 5.74-5.65 (m, 1H), 3.61 (q, J=6.8 Hz, 1H), 3.47-3.35 (m, 1H), 3.06-2.93 (m, 1H), 2.27 (d, J=12.8 Hz, 1H), 1.89-1.79 (m, 1H), 1.67-1.56 (m, 3H), 1.42 (d, J=11.2 Hz, 1H), 0.98 (t, J=7.6 Hz, 3H)

### Example 3: Preparation of Key Intermediate III-1

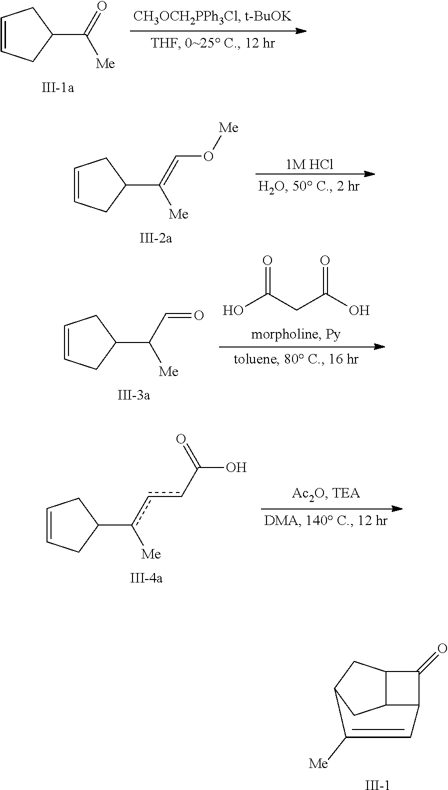

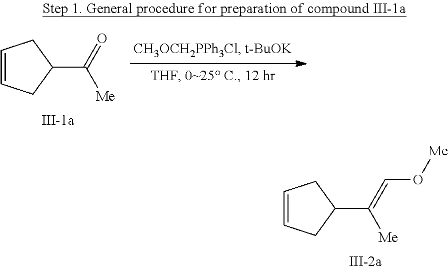 [SMILES: CC(=O)C1CC=CC1 ; CO/C=C(\C)C1CC=CC1]

To a solution of methoxymethyl(triphenyl)phosphonium chloride (7.49 g, 21.8 mmol) in THF (100 mL) was added t-BuOK (1 M, 36.31 mL) at 0° C. under N₂. The mixture was stirred at 0° C. for 0.5 h, then compound III-1a (2 g, 18.2 mmol) was added, the mixture was stirred at 25° C. for 12 h. TLC (PE:EtOAc=10:1) showed a new spot was formed. The mixture was used into next step without further purification.

### Step 2. General Procedure for Preparation of Compound III-3a.

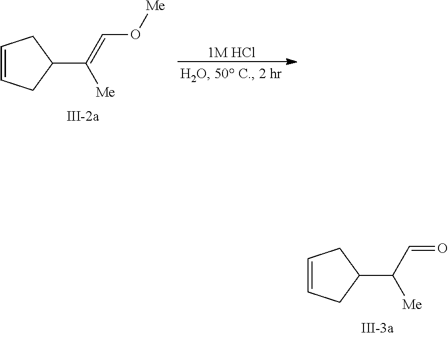

To the mixture of compound III-2a was added HCl (5 mL, 1M) was heated to 50° C. and stirred for 2 h. TLC (PE:EA=10:1) showed the starting material was consumed, a new spot was formed. The mixture was extracted with EtOAc (20 mL×2), the organic layer was washed with brine, dried over Na₂SO₄, filtered and concentrated under reduced pressure. The residue was purified by flash silica gel chromatography (20 g Silica Flash Column, Eluent of 0˜5% Ethyl acetate/commercial hexanes gradient @40 mL/min) to give III-3a (720 mg, 32% yield) as yellow oil.

### Step 3. General Procedure for Preparation of Compound III-4a.

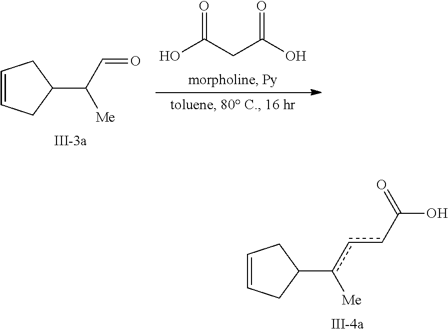

To a solution of compound III-3a (720 mg, 5.8 mmol) in Toulene (12 mL), was added Pyridine (1.05 g, 13.34 mmol), malonic acid (905 mg, 8.70 mmol), and morpholine (303 mg, 3.48 mmol), the mixture was stirred at 85° C. for 16 h. LCMS showed the starting material was consumed, the desired mass was detected. TLC (PE:EtOAc=5:1) showed a new spot was formed. The reaction mixture was diluted with H2O (50 mL) and extracted with EtOAc (80 mL×3). The combined organic layers were washed with brine (40 mL), dried over Na₂SO₄, filtered and concentrated under reduced pressure. The residue was purified by column on silica gel eluted with (Commercial hexanes:Ethyl acetate=10:0 to 5:1) to give III-4a (700 mg, 73% yield) as yellow oil

LC-MS: m/z: 165.0, M−H⁺

### Step 4. General Procedure for Preparation of Compound III-1.

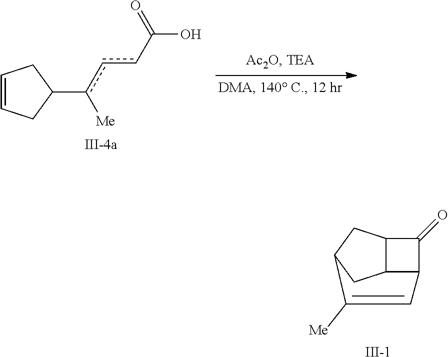

To a solution of compound III-4a (245 mg, 1.47 mmol) in DMA (12 mL) was added Ac₂O (271 mg, 2.65 mmol) and TEA (164 mg, 1.62 mmol), the mixture was stirred at 140° C. for 12 h under N₂. LCMS showed the starting material was consumed, the desired mass was detected. The mixture was diluted with H₂O (30 mL), the mixture was extracted with EtOAc (40 mL), the organic layer was washed with brine (20 mL×3), dried over Na₂SO₄, filtered and concentrated under reduced pressure (25° C.). The residue was purified by flash silica gel chromatography (12 g Silica Flash Column, Eluent of 0˜2% Ethylacetate/Commercial hexanes@30 mL/min) to give compound III-1 (165 mg, 38% yield) as brown oil.

LC-MS: m/z: 149.1, M+H⁺

### Example 4: Synthesis of Compound I

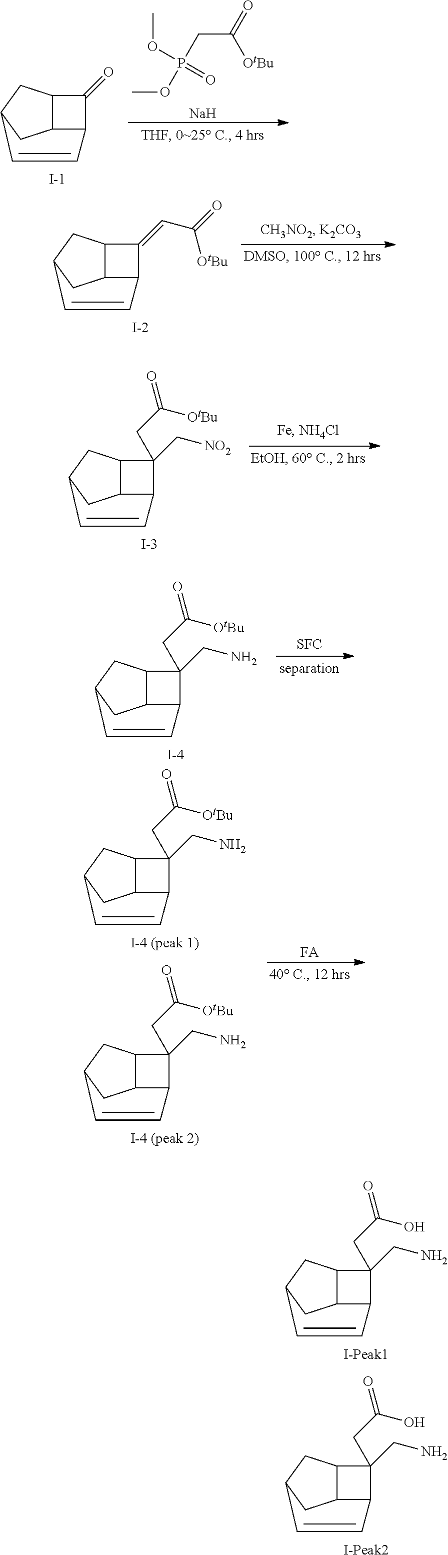

### Step 1. General Procedure for Preparation of Compound I-2.

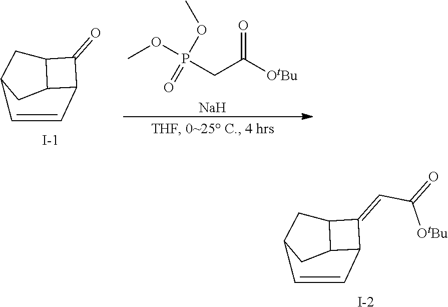

To a solution of tert-butyl 2-dimethoxyphosphorylacetate (1.40 g, 6.26 mmol, 1.24 mL) in THF (28 mL) was added NaH (250.40 mg, 6.26 mmol, 60% purity) at 0° C. under N₂, the mixture was stirred at 0° C. for 0.5 h, then I-1 (0.70 g, 5.22 mmol) in THF (7 mL) was added dropwise at 0° C. under N₂, the mixture was stirred at 20° C. for 1 h. After the reaction was completed, the mixture was quenched ice water (100 mL), extracted with MTBE (50 mL×3), the organic phase was washed with sat NH₄Cl (50 mL), brine (50 mL×3), dried over Na₂SO₄ and concentrated under reduced pressure. The residue was purified by column chromatography (SiO₂, Petroleum ether:Ethyl acetate=20:1, Rf (P1)=0.5.) to give compound 1-2 (0.86 g, 70.9% yield) was obtained as a colorless solid.

LC-MS: m/z=177.2, M-56

¹H NMR: (400 MHz, CDCl₃)

δ=6.42-6.30 (m, 1H), 6.03-5.70 (m, 1H), 5.40-5.24 (m, 1H), 3.93-3.62 (m, 1H), 3.40-3.17 (m, 1H), 3.04 (d, J=4.0 Hz, 1H), 2.81-2.71 (m, 1H), 2.05-1.84 (m, 2H), 1.68-1.59 (m, 1H), 1.47 (d, J=6.4 Hz, 9H), 1.42-1.33 (m, 1H).

### Step 2. General Procedure for Preparation of Compound I-3.

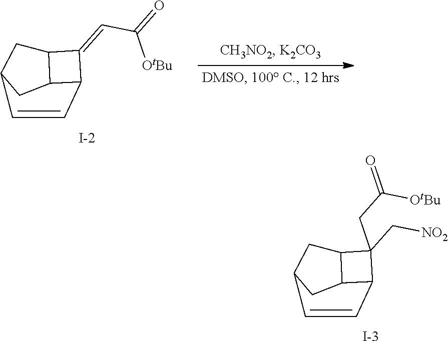

To a solution of I-2 (0.86 g, 3.70 mmol) in DMSO (11 mL) was added K₂CO₃ (2.05 g, 14.8 mmol) and CH₃NO₂ (2.26 g, 37.0 mmol, 2.00 mL) at 20° C., the mixture was stirred at 100° C. at 12 h under N₂. After the reaction was completed and cooled to room temperature, the mixture was diluted with water (50 mL), extracted with MTBE (25 mL×5), the organic phase was washed with brine (25 mL×3), dried over Na₂SO₄ and concentrated under reduced pressure. The residue was purified by column chromatography (SiO₂, Petroleum ether:Ethyl acetate=5:1, Rf (P1)=0.5.) to give compound 1-3 (0.8 g, 73.6% yield) was obtained as a colorless oil.

LC-MS: m/z=316.3, M+Na⁺

¹H NMR: (400 MHz, CDCl₃)

δ=6.53-6.42 (m, 1H), 5.62 (dd, J=6.4, 9.2 Hz, 1H), 5.02-4.80 (m, 2H), 3.17-3.02 (m, 1H), 2.87-2.78 (m, 1H), 2.77-2.67 (m, 1H), 2.65-2.58 (m, 1H), 2.51-2.39 (m, 2H), 1.73-1.60 (m, 2H), 1.55-1.50 (m, 1H), 1.45 (s, 9H), 1.30 (d, J=11.2 Hz, 1H)

### Step 3. General Procedure for Preparation of Compound I-4.

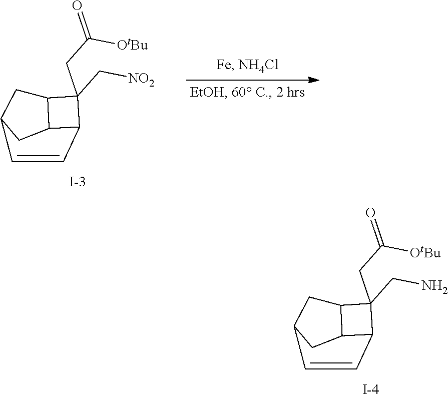

To a solution of I-3 (630 mg, 2.15 mmol) in EtOH (12 mL) and H₂O (5.4 mL) was added Fe (839 mg, 15.0 mmol) and NH₄Cl (344 mg, 6.44 mmol), the mixture was stirred at 80° C. for 2 h. After the reaction was completed and cooled to room temperature, the mixture was filtered and the filtrated was diluted with water (10 mL), extracted with EtOAc (10 mL×3), the organic layer was washed with brine, dried over Na₂SO₄, filtered and concentrated under reduced pressure. The residue was purified by prep-HPLC (column: CD01-Phenomenex luna C18 150*25 mm*10 μm; mobile phase: [H2O (0.225% FA)−ACN]; gradient:15%-45% B over 10.0 min) to give 1-4 (420 mg, 74.6% yield) was obtained as a white solid.

LC-MS: m/z: 264.2, M+H⁺

¹H NMR:(400 MHz, MeOD-d₄)

δ=8.52 (s, 1H), 6.49 (dd, J=7.6, 9.2 Hz, 1H), 5.61 (dd, J=6.4, 9.2 Hz, 1H), 3.43-3.33 (m, 2H), 3.15-3.07 (m, 1H), 2.75-2.69 (m, 1H), 2.69-2.63 (m, 1H), 2.51-2.34 (m, 3H), 1.76-1.68 (m, 1H), 1.67-1.60 (m, 1H), 1.58-1.52 (m, 1H), 1.48-1.41 (m, 9H), 1.31-1.24 (m, 1H)

### Step 4. General Procedure for Preparation of Compound I-4(peak1&peak2).

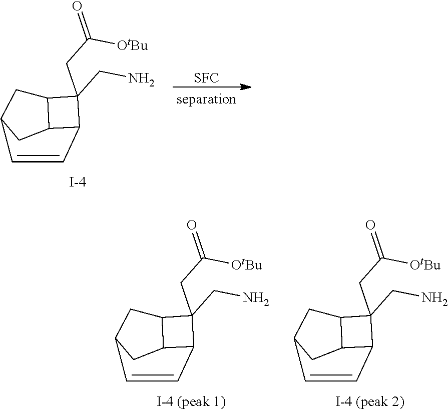

The I-4 (420 mg) was separated by SFC (column: Chiral-DAICEL CHIRALCEL OX (250 mm*30 mm, 10 um); mobile phase: [CO₂-EtOH (0.1% Et₂NH)]; B %:15%, isocratic elution mode) to give the 1-4 (peak1) (150 mg, 32% yield) and 1-4 (peak2) (138 mg, 28% yield).

### Step 5. General Procedure for Preparation of Compound I-Peak1.

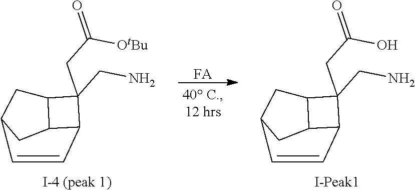

To a solution of I-4 (peak1) (150 mg, 569 μmol) in formic acid (2 mL) was stirred at 40° C. for 12 h. After the reaction was completed, the reaction mixture was concentrated under reduced pressure. The residue was purified by prep-HPLC (column: CD01-Phenomenex luna C18 150*25 mm*10 um; mobile phase: [H₂O (0.225% FA)−ACN]; gradient:0%-20% B over 25.0 min) to give compound I-Peak1 (60 mg, 50.8% yield) as a white solid.

LC-MS: Rt=1.408 min, m/z=208.2, M+1

SFC: Ret Time: 1.842 min,

Optical Rotation: [α]=129.27°, 25.0° C., H₂O

¹H NMR: (400 MHz, D₂O)

δ=6.46 (t, J=8.4 Hz, 1H), 5.64-5.53 (m, 1H), 3.38-3.24 (m, 2H), 3.03 (d, J=4.0 Hz, 1H), 2.71-2.62 (m, 1H), 2.59-2.50 (m, 1H), 2.44-2.29 (m, 3H), 1.67-1.52 (m, 2H), 1.50-1.41 (m, 1H), 1.22-1.13 (m, 1H)

### Step 6. General Procedure for Preparation of Compound I-Peak2.

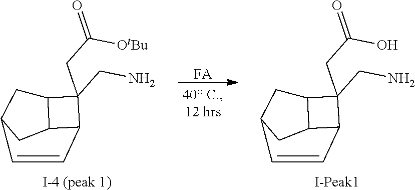

To A solution of I-4 (peak2) (138 mg, 523 μmol) in formic acid (2 mL) was stirred at 40° C. for 12 h. After the reaction was completed, the reaction mixture was concentrated under reduced pressure. The residue was purified by prep-HPLC (column: CD01-Phenomenex luna C18 150*25 mm*10 μm; mobile phase: [H₂O (0.225% FA)−ACN]; gradient:0%-20% B over 25.0 min) to give compound I-Peak2 (74 mg, 68% yield) as a white solid.

LC-MS: Rt=1.477 min, m/z=208.2, M+1

SFC: Ret Time: 1.815 min

Optical Rotation: [α]=−121.24°, 25.0° C., H₂O

¹H NMR: (400 MHz, D2O)

δ=6.46 (t, J=8.4 Hz, 1H), 5.58 (dd, J=6.4, 9.2 Hz, 1H), 3.39-3.23 (m, 2H), 3.03 (d, J=3.6 Hz, 1H), 2.70-2.62 (m, 1H), 2.58-2.49 (m, 1H), 2.45-2.29 (m, 3H), 1.68-1.52 (m, 2H), 1.51-1.42 (m, 1H), 1.17 (d, J=11.2 Hz, 1H)

### Example 5: Synthesis of Compound II

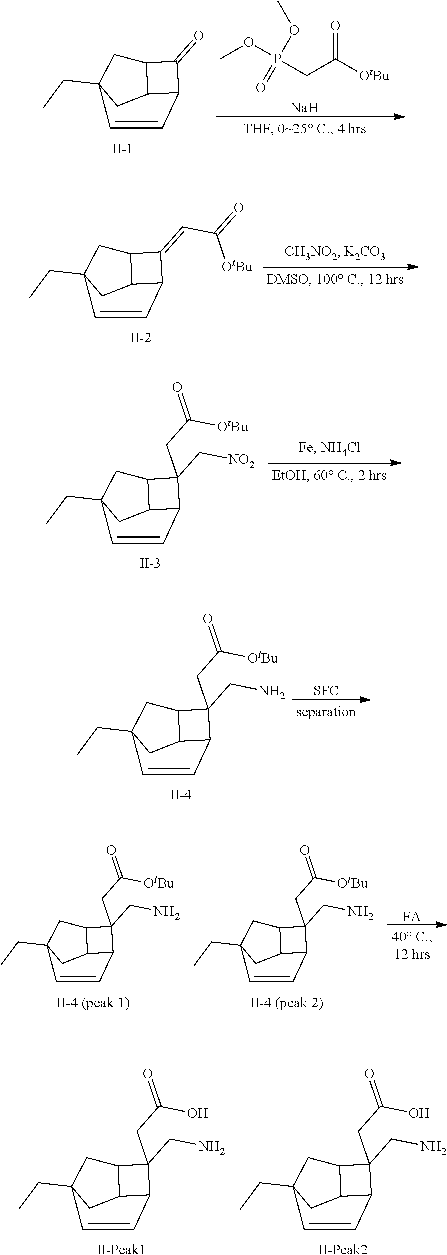

### Step 1. General Procedure for Preparation of Compound II-2.

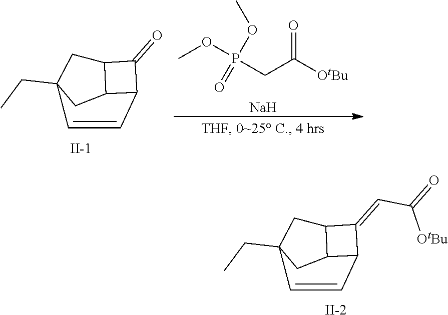

To a solution of tert-butyl 2-dimethoxyphosphorylacetate (995.00 mg, 4.44 mmol) in THF (6 mL) was added NaH (222 mg, 5.55 mmol, 60% purity) under N₂ at 0° C. The reaction mixture was stirred for 0.5 h at 0° C. and then compound II-1 (600 mg, 3.70 mmol) in THF (6 mL) was added to the mixture. The reaction mixture was stirred for 4 h at 25° C. LCMS showed the starting material was consumed, the desired mass was detected. The mixture was quenched by sat. NH₄Cl (10 mL), the organic layer was extracted with EtOAc (20 mL×2), dried over Na₂SO₄, filtered and concentrated under reduced pressure. The residue was purified by flash silica gel chromatography (12 g Silica flash column, eluent of 0˜5% Ethyl acetate/Petroleum ether gradient 25 mL/min) to give compound 11-2 (470 mg, 49% yield) as colorless oil.

¹H NMR: (400 MHz, CDCl₃)

δ=6.25-6.12 (m, 1H), 6.05-5.75 (m, 1H), 5.41-5.25 (m, 1H), 3.89-3.68 (m, 1H), 3.38-3.19 (m, 1H), 3.10-2.97 (m, 1H), 2.00-1.84 (m, 1H), 1.80-1.66 (m, 1H), 1.60-1.53 (m, 2H), 1.51-1.45 (m, 1OH), 1.38-1.26 (m, 1H), 0.97 (dt, J=2.8, 7.6 Hz, 3H)

### Step 2. General Procedure for Preparation of Compound 11-3.

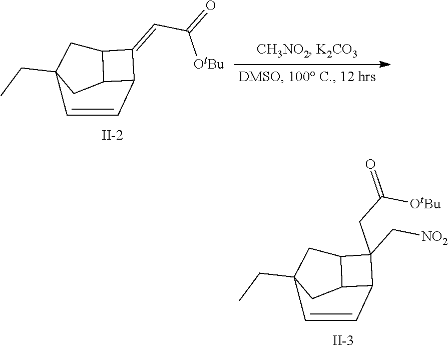

To a solution of compound 11-2 (470 mg, 1.81 mmol) and K₂CO₃ (748 mg, 5.42 mmol) in DMSO (5 mL) were added CH₃NO₂ (1.10 g, 18.05 mmol) at 25° C. under N₂. The reaction mixture was stirred for 16 h at 100° C. LCMS showed the starting material was consumed, and the desired mass was detected. The mixture was cooled to 25° C. and diluted with water (10.00 mL), extracted with EtOAc (20.00 mL×2). Then the combined organic phase was washed with brine (20.0 mL), dried over Na₂SO₄, filtered and concentrated. The residue was purified by flash silica gel chromatography (12 g Silica Flash Column, Eluent of 0˜5% Ethyl acetate/Petroleum ether gradient 25 mL/min) to give compound 11-3 (500 mg, 86% yield) as colorless oil.

¹H NMR: (400 MHz, CDCl₃)

δ=6.28 (d, J=9.2 Hz, 1H), 5.66 (dd, J=6.4, 9.2 Hz, 1H), 4.97-4.76 (m, 2H), 3.15-3.05 (m, 1H), 2.88-2.78 (m, 1H), 2.68-2.58 (m, 1H), 2.54-2.33 (m, 2H), 1.68 (d, J=13.6 Hz, 1H), 1.57-1.52 (m, 2H), 1.48-1.43 (m, 1OH), 1.39-1.33 (m, 1H), 1.28-1.19 (m, 1H), 0.97 (t, J=7.6 Hz, 3H)

### Step 3. General Procedure for Preparation of Compound 11-4.

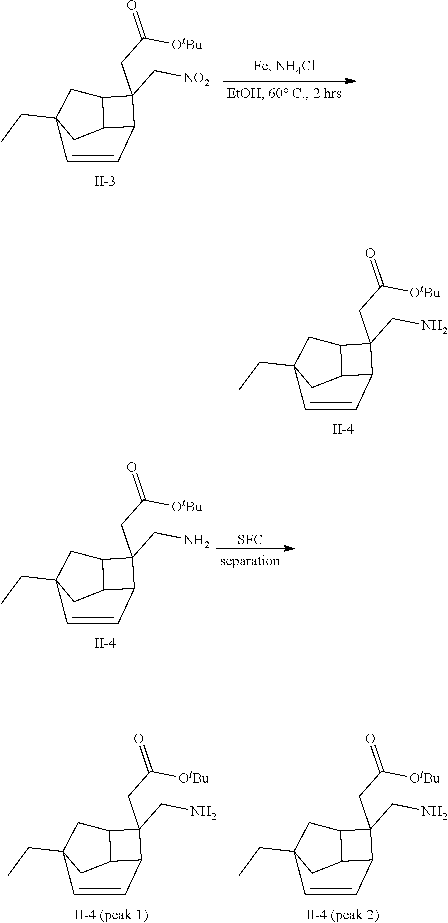

To a solution of compound 11-3 (500 mg, 1.56 mmol) in EtOH (5 mL) and H₂O (1.25 mL) was degassed and purged with N₂ for 3 times, and then was slowly added Fe (434 mg, 7.78 mmol) and NH₄Cl (666 mg, 12.45 mmol) under N₂ atmosphere at 25° C. Then the mixture was stirred at 55° C. for 2 hrs. under N₂ atmosphere. LCMS showed the starting material was consumed, and the desired mass was detected. The mixture was filtered and the filtrated was extracted with EtOAc (25 mL×2), the organic layer was washed with brine, dried over Na₂SO₄, filtered and concentrated under reduced pressure.

The residue was separated by SFC (column: Chiral-IG-30-Daicel ChiralPak IG (250*30 mm, 10 um); mobile phase: [Heptane-EtOH (0.1% IPA)]; B %: 6%, isocratic elution mode) to give compound II-4-Peak1(130 mg, 29% yield) as yellow oil and compound II-4-Peak2 (180 mg, 40% yield) as yellow solid

II-4-Peak1:

SFC: Rt=2.118 min

II-4-Peak2:

SFC: Rt=3.818 min

### Step 4. General Procedure for Preparation of Compound II-Peak1/II-Peak2.

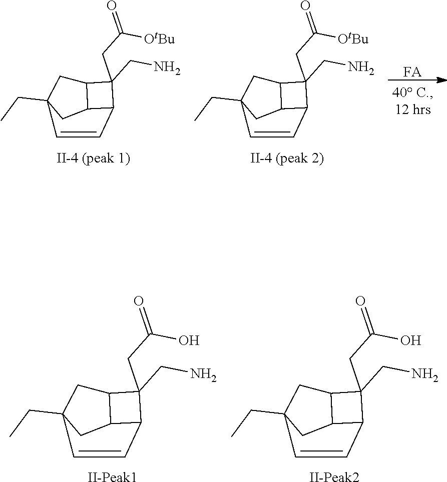

A solution of compound II-4-Peak1 (180 mg, 0.62 mmol) in formic acid (3 mL) was stirred for 16 h at 40° C. LCMS showed the starting material was consumed, the desired mass was detected. The mixture was concentrated under reduced pressure. The residue was purified by prep-HPLC (column: CD01-Phenomenex Luna C18 150*25 mm*10 μm; mobile phase: [H₂O (0.225% FA)−ACN]; gradient:5%-35% B over 10.0 min) to give compound II-Peak1 (95.7 mg, 66% yield) as white solid.

SFC: Rt=4.228 min

Optical Rotation: [α]=277.31°, 25° C., MeOH

¹H NMR: (400 MHz, D₂O)

δ=6.31 (d, J=9.2 Hz, 1H), 5.65 (dd, J=6.0, 9.2 Hz, 1H), 3.36-3.21 (m, 2H), 3.12-2.99 (m, 1H), 2.63-2.51 (m, 1H), 2.46-2.32 (m, 3H), 1.65 (d, J=13.6 Hz, 1H), 1.57-1.40 (m, 2H), 1.39-1.24 (m, 2H), 1.12 (d, J=11.2 Hz, 1H), 0.89 (t, J=7.6 Hz, 3H)

A solution of compound II-4-Peak2 (170 mg, 0.50 mmol,) in formic acid (2.5 mL) was stirred for 16 h at 40° C. LCMS showed the starting material was consumed, the desired mass was detected. The mixture was concentrated under reduced pressure. The residue was purified 7 by prep-HPLC(column: CD01-Phenomenex Luna C18 150*25 mm*10 um; mobile phase: [H₂O (0.225% FA)−ACN]; gradient:5%-35% B over 10.0 min) to give compound II-Peak2 (98.87 mg, 420.15 μmol, 83.40% yield) as white solid.

SFC: Rt=3.557 min.

Optical Rotation: [α]=−151.16°, 25° C., MeOH

¹H NMR: (400 MHz, D₂O)

δ=6.33 (d, J=9.2 Hz, 1H), 5.66 (t, J=7.2 Hz, 1H), 3.37-3.21 (m, 2H), 3.05 (d, J=1.2 Hz, 1H), 2.61-2.52 (m, 1H), 2.48-2.36 (m, 3H), 1.66 (d, J=13.6 Hz, 1H), 1.54-1.44 (m, 2H), 1.40-1.28 (m, 2H), 1.14 (d, J=10.8 Hz, 1H), 0.90 (br t, J=7.2 Hz, 3H)

### Example 6: Synthesis of Compound III

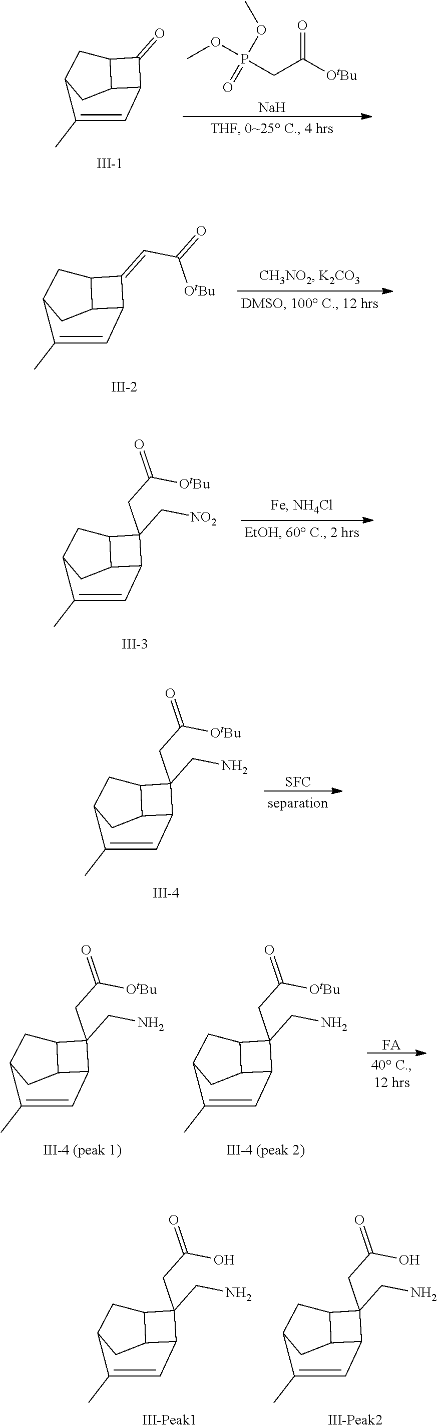

### Step 1. General Procedure for Preparation of Compound III-2.

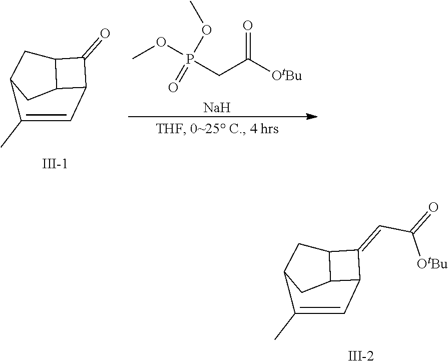

To a solution of tert-butyl 2-dimethoxyphosphorylacetate (299 mg, 1.34 mmol) in THF (3 mL) was added NaH (67 mg, 1.67 mmol, 60% purity) under N₂ at 0° C. The reaction mixture was stirred for 0.5 h at 0° C. and then compound II-1 (165 mg, 1.11 mmol) in THF (3 mL) was added to the mixture. The reaction mixture was stirred for 4 h at 25° C. LCMS showed the starting material was consumed, and the desired mass was detected. The mixture was quenched by sat. NH₄Cl (10 mL), the organic layer was extracted with EtOAc (20 mL×2), dried over Na₂SO₄, filtered and concentrated under reduced pressure. The residue was purified by flash silica gel chromatography (12 g Silica Flash Column, Eluent of 0˜2% Ethylacetate/commercial hexanes 30 mL/min) to give compound III-2 (180 mg, 66% yield) as colorless oil.

LC-MS: EW58822-263-P1A1, Rt=0.719 min, m/z=191.1, M-56+H⁺

### Step 2. General Procedure for Preparation of Compound III-3.

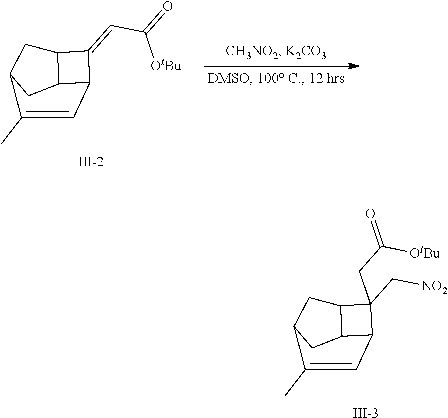

To a solution of compound 11-2 (470 mg, 1.81 mmol) and K₂CO₃ (748 mg, 5.42 mmol) in DMSO (5 mL) were added CH₃NO₂ (1.10 g, 18.05 mmol) at 25° C. under N₂. The reaction mixture was stirred for 16 h at 100° C. LCMS showed the starting material was consumed, and the desired mass was detected. The mixture was cooled to 25° C. and diluted with water (10.00 mL), extracted with EtOAc (20.00 mL×2). Then the combined organic phase was washed with brine (20.0 mL), dried over Na₂SO₄, filtered and concentrated. The residue was purified by flash silica gel chromatography (12 g Silica Flash Column, Eluent of 0˜5% Ethyl acetate/Petroleum ether gradient 25 mL/min) to give compound 11-3 (500 mg, 86% yield) as colorless oil.

### Step 3. General Procedure for Preparation of Compound III-4

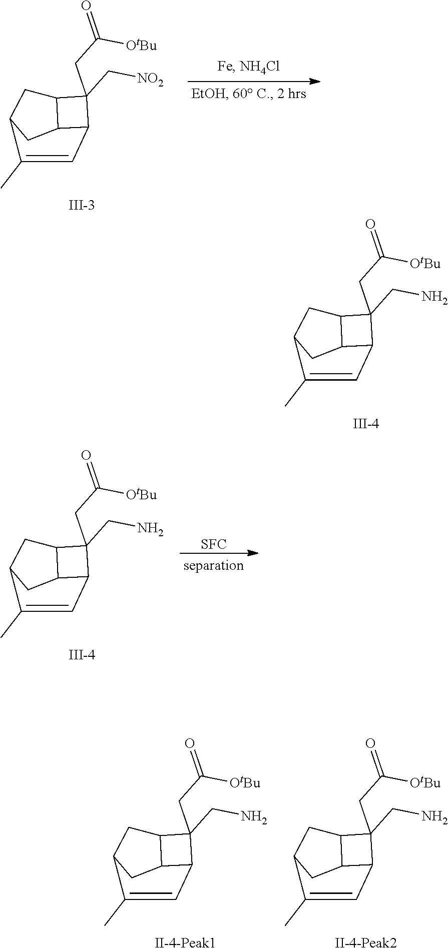

To a solution of compound III-3 (150 mg, 0.49 mmol) in EtOH (3 mL) and H₂O (0.75 mL) was degassed and purged with N₂ for 3 times, and then was slowly added Fe (136 mg, 2.44 mmol) and NH₄Cl (209 mg, 3.90 mmol) under N₂ atmosphere at 25° C. Then the mixture was stirred at 55° C. for 2 hrs under N₂ atmosphere. LCMS showed the starting material was consumed, the desired mass was detected. The mixture was filtered and the filtrated was extracted with EtOAc (25 mL×2) and H₂O (10 mL), the organic layer was washed with brine, dried over Na₂SO₄, filtered and concentrated under reduced pressure.

The residue was separated by SFC(column: Chiral-Cellulose-2-30-Phenomenex-Cellulose-2 (250 mm*30 mm, 10 um; mobile phase: [CO₂-EtOH (0.1% NH₃H2O)]; B %:25%, isocratic elution mode) to give compound III-4-Peak1 (50 mg, 37% yield) as yellow oil and compound III-4-Peak2(45 mg, 34% yield) as yellow oil.

III-4-Peak1:

LC-MS: Rt=0.397 min, m/z=278.3, M+H⁺

SFC: Rt=1.302 min

III-4-Peak2:

LC-MS: Rt=0.403 min, m/z=278.3, M+H⁺

SFC: Rt=1.524 min

### Step 3. General Procedure for Preparation of Compound Ill.

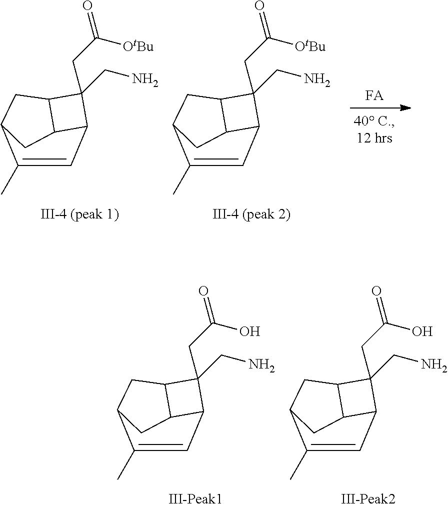 [SMILES: CC1=CC2C3CC1CC3C2(CN)CC(=O)OC(C)(C)C ; CC1=CC2C3CC1CC3C2(CN)CC(=O)OC(C)(C)C ; "FA" ; CC1=CC2C3CC1CC3C2(CN)CC(=O)O ; CC1=CC2C3CC1CC3C2(CN)CC(=O)O]

A solution of compound III-4-Peak1 (50 mg, 0.18 mmol) in FORMIC ACID (1 mL) was heated to 40° C. for 16 h. LCMS showed the starting material was consumed, the desired mass was detected. The mixture was concentrated under reduced pressure to give compound III-Peak1 (25.49 mg, 64% yield) as yellow solid, which was without purification.

SFC: Ret Time:1.248 min

LC-MS: Rt=1.210 min, m/z=222.3, M+H⁺

OR: [α]=44.51°, 25° C., MeOH

¹H NMR: (400 MHz, D₂O)

δ=5.27 (d, J=6.0 Hz, 1H), 3.41-3.27 (m, 2H), 3.08-2.97 (m, 1H), 2.54-2.48 (m, 2H), 2.33 (s, 3H), 1.82 (d, J=0.8 Hz, 3H), 1.66-1.57 (m, 2H), 1.49 (td, J=4.0, 11.6 Hz, 1H), 1.21 (d, J=11.6 Hz, 1H)

A solution of compound III-4-Peak2 (45 mg, 0.16 mmol) in FORMIC ACID (1 mL) was heated to 40° C. for 16 h. LCMS showed the starting material was consumed, the desired mass was detected. The mixture was concentrated under reduced pressure to give compound III-Peak2 (25.49 mg, 64% yield) (18.04 mg, 50% yield) as off-white solid, which was without purification.

SFC: Ret Time:1.277 min

LC-MS: Rt=1.849 min, m/z=222.2, M+H⁺

OR: [α]=−46.83°, 25° C., MeOH

¹H NMR: (400 MHz, D₂O) 5=5.26 (d, J=6.0 Hz, 1H), 3.38-3.26 (m, 2H), 3.06-2.96 (m, 1H), 2.53-2.45 (m, 2H), 2.43-2.26 (m, 3H), 1.82 (d, J=1.2 Hz, 3H), 1.71-1.57 (m, 2H), 1.49 (td, J=4.0, 11.2 Hz, 1H), 1.20 (d, J=11.2 Hz, 1H).

### Example 7: Preparation of Compound IV

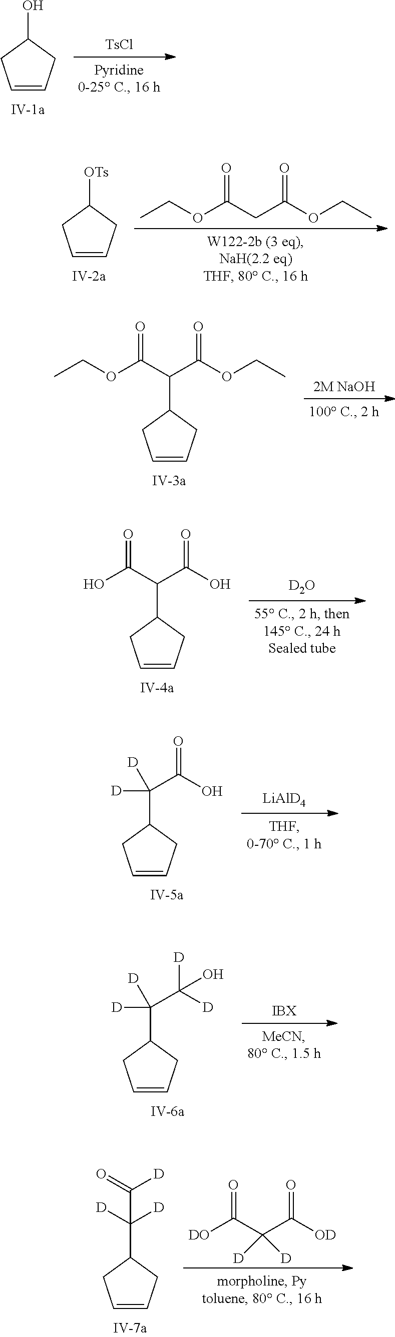

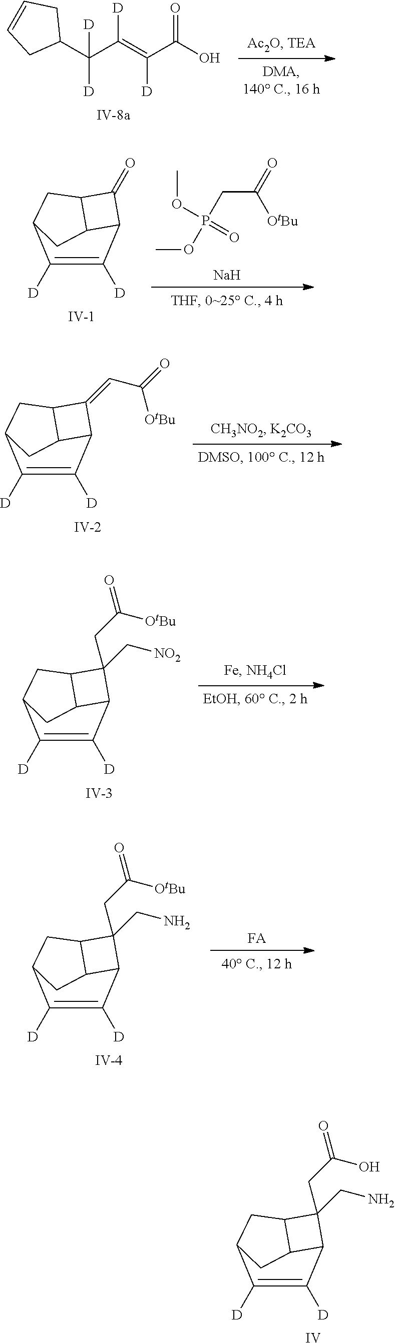

### Step-1: Preparation of Intermediate IV-1a

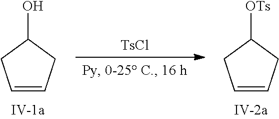 [SMILES: OC1CC=CC1 ; "TsCl" ; Cc1ccc(S(=O)(=O)OC2CC=CC2)cc1]

To a solution of compound IV-1a (15 g, 178 mmol) in Pyridine (75 mL) was added Ts-CI (40.8 g, 214 mmol) at 0° C., the reaction mixture was stirred for 16 hrs at 20° C. TLC (PE:EtOAc=10:1, Rf=0.3) showed the starting material was consumed, and a new spot was formed. The mixture was poured into 4 M HCl (300 mL) and stirred for 0.5 hr at 25° C. The mixture was extracted with EtOAc (200 mL×2), the organic layer was washed with brine (200 mL), dried over Na₂SO₄, filtered and concentrated under reduced pressure. The residue was triturated with hexane (80 mL), the suspension was filtered and the solid was dried under reduced pressure to give IV-2a (26.5 g, 62% yield) as an off-white solid.

### Step-2: Preparation of Intermediate IV-3a

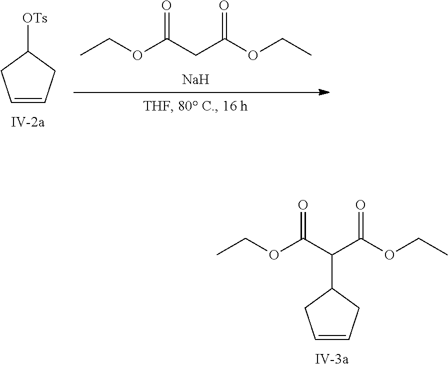

To a solution ethyl malonate (42.0 g, 262 mmol) in THF (420 mL) was added NaH (9.23 g, 231 mmol) at 0° C. under N₂. The reaction mixture was stirred for 0.5 hr at 0° C. and then compound IV-2a (25.0 g, 105 mmol) in THF (100 mL) was added to the mixture. The reaction mixture was stirred for 16 hrs at 80° C. The reaction was quenched with saturated aqueous NH₄Cl (100 mL). The mixture was extracted with EtOAc (200 mL×2), the organic layer was washed with brine (100 mL), dried over Na₂SO₄, filtered and concentrated under reduced pressure. The residue was purified by column chromatography (80.0 g Silica Flash Column, Eluent of 0˜2% Ethyl acetate/Commercial hexanes gradient 40 mL/min) to give compound IV-3a (20.5 g, 86% yield) as colorless oil.

¹H NMR: (400 MHz, CD₃Cl) δ 5.67 (s, 2H), 4.20 (q, J=7.2 Hz, 4H), 3.35 (d, J=10.0 Hz, 1H), 3.03-2.86 (m, 1H), 2.58 (dd, J=8.8, 14.4 Hz, 2H), 2.15 (dd, J=6.8, 14.4 Hz, 2H), 1.27 (t, J=7.2 Hz, 6H).

### Step-3: Preparation of Intermediate IV-4a

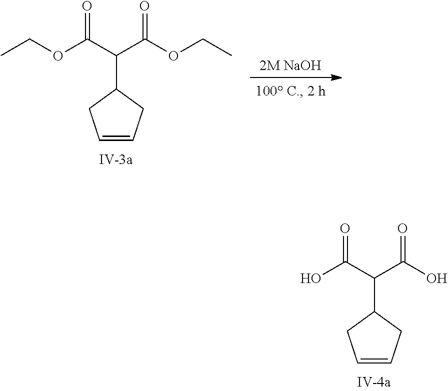 [SMILES: CCOC(=O)C(C(=O)OCC)C1CC=CC1 ; "2M NaOH" ; O=C(O)C(C(=O)O)C1CC=CC1]

A solution of compound IV-3a (16.0 g, 70.7 mmol) in NaOH in H₂O (128 mL, 2M) was heated to 100° C. and stirred for 2 hrs. TLC (PE:EtOAc=10:1, Rf=0) showed the starting material was consumed, a new spot was formed. HCl (12 M) was added to the mixture until the solid was formed at 0° C. The suspension was filtered and solid was dried under reduced pressure to give compound IV-4a (10.0 g, 83% yield) as white solid, which was used in the next step without further purification.

### Step-4: Preparation of Intermediate IV-5a

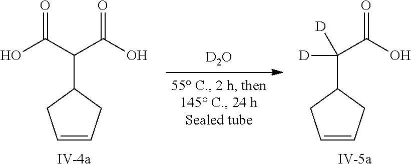 [SMILES: O=C(O)C(C(=O)O)C1CC=CC1 ; "D2O" ; [2H]C([2H])(C(=O)O)C1CC=CC1]

A solution of compound IV-4a (1.00 g, 5.88 mmol) in D₂O (18 mL) was heated to 55° C. for 2 hrs in 50 mL autoclave by 5 batch and then heated to 145° C. and stirred for 24 hrs. TLC (PE:EtOAc=5:1, Rf=0.3) showed the starting material was consumed, a new spot was formed. The mixture was cooled to 25° C. and then extracted with EtOAc (40 mL×2), the organic layer was washed with brine (20 mL), dried over Na₂SO₄, filtered and concentrated under reduced pressure to give compound IV-5a (2.20 g, 58.0% yield) as brown oil, which was used in the next step without further purification.

¹H NMR: (400 MHz, CD₃Cl) δ 5.68 (s, 2H), 2.57-2.55 (m, 1H), 2.70-2.53 (m, 3H), 2.11-2.06 (m, 1H), 2.03 (s, 1H).

### Step-5: Preparation of Intermediate IV-6a

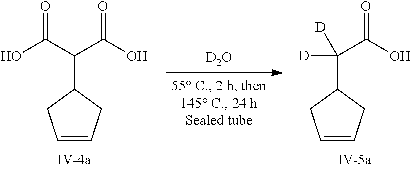 [SMILES: O=C(O)C(C(=O)O)C1CC=CC1 ; "D2O" ; [2H]C([2H])(C(=O)O)C1CC=CC1]

To a solution of compound IV-5a (2.20 g, 17.2 mmol) in THF (44 mL) was added LiAlD₄ (1.30 g, 34.3 mmol) at 0° C. under N₂, then the mixture was stirred at 70° C. for 1 hr under N₂. GCMS showed the starting material was consumed, the desired mass was detected. TLC (PE:EtOAc=5:1, Rf=0.28) showed the starting material was consumed, a new spot was formed. The mixture was quenched by Na₂SO₄·10H₂O (20 g), the suspension was filtered and filtrated was concentrated under reduced pressure to give compound IV-6a (1.75 g, 88% yield) as yellow oil, which was used into next step without purification.

GCMS: Ret Time: 4.241 min

¹H NMR: (400 MHz, CD₃Cl) δ 5.71-5.61 (m, 2H), 2.72-2.52 (m, 3H), 2.05 (dd, J=5.2, 13.6 Hz, 2H)

### Step-6: Preparation of Intermediate IV-7a

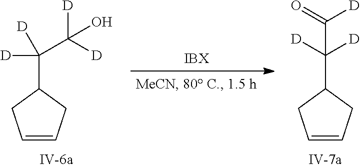

To a solution of compound IV-6a (1.75 g, 15.1 mmol) in MeCN (30 mL) was added IBX (6.33 g, 22.6 mmol) at 25° C. The reaction was heated to 80° C. for 1.5 hrs. TLC (PE:EtOAc=5:1, Rf=0.7) showed the starting material was consumed, a new spot was formed. The mixture was cooled to 25° C. and filtered, the filtrated was concentrated under reduced pressure to give compound IV-7a (1.50 g, 88% yield) as yellow oil, which was used in the next step without further purification.

### Step-7: Preparation of Intermediate IV-8a

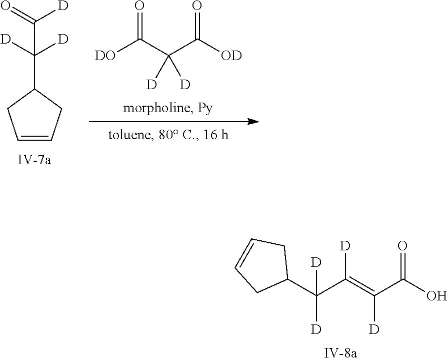

To a solution of compound IV-7a (1.50 g, 13.3 mmol) in toluene (18 mL) was added Pyridine (2.41 g, 30.5 mmol), morpholine (693 mg, 7.95 mmol) and malonic-d2 acid-d2 (2.15 g, 19.9 mmol) at 25° C. The reaction mixture was stirred for 16 hrs at 80° C. LCMS showed the starting material was consumed, and the desired mass was detected. The pH of the reaction mixture was adjusted to 12 by using 2M NaOH and extracted with EtOAc (20 mL×2). The pH of the water phase was adjusted to 3 by using 1 M HCl, the mixture was extracted with EtOAc(40 mL×2). The combined organic layers were washed with brine (100 mL), dried over Na₂SO₄, filtered and concentrated under reduced pressure. The residue was purified by flash silica gel chromatography (20 g Silica Flash Column, Eluent of 0˜8% Ethyl acetate/Commercial hexanes gradient 30 mL/min) to compound IV-8a (1.08 g, 52% yield) as colorless oil.

LC-MS: m/z=155.3, M−H⁺

¹H NMR: (400 MHz, CD₃CI) 6 5.72-5.63 (m, 2H), 2.53 (dd, J=8.4, 14.0 Hz, 2H), 2.45-2.26 (m, 1.5H), 2.07-1.97 (m, 2H).

### Step-8: Preparation of Intermediate IV-1

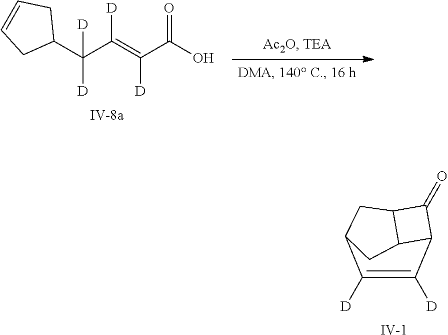

To a solution of compound IV-8a (320 mg, 2.05 mmol) in DMA (15 mL) was added Ac₂O (376 mg, 3.69 mmol) and Et₃N (228 mg, 2.25 mmol) at 25° C. (in 4 batches). The reaction mixture was stirred for 16 hrs at 140° C. LCMS showed the starting material was consumed, the desired mass was detected. The mixture was diluted with H₂O (30 mL), the mixture was extracted with EtOAc (40 mL), the organic layer was washed with brine (20 mL×3), dried over Na₂SO₄, filtered and concentrated under reduced pressure (25° C.). The residue was purified by flash silica gel chromatography (12 g Silica Flash Column, Eluent of 0˜2% EtOAc/commercial hexanes 30 mL/min) to give compound IV-9b (520 mg, 51% yield) as yellow solid.

LC-MS: m/z=137.2, M+H⁺

¹H NMR: (400 MHz, CD₃Cl) δ 6.45 (d, J=7.6 Hz, 0.5H), 3.64 (t, J=6.8 Hz, 1H), 3.47-3.37 (m, 1H), 2.99 (q, J=5.6 Hz, 1H), 2.87-2.74 (m, 1H), 2.28 (d, J=12.8 Hz, 1H), 2.08 (ddd, J=5.2, 10.8, 12.8 Hz, 1H), 1.79 (td, J=4.0, 11.2 Hz, 1H), 1.46 (d, J=11.2 Hz, 1H)

### Step-9: Preparation of Intermediate IV-2

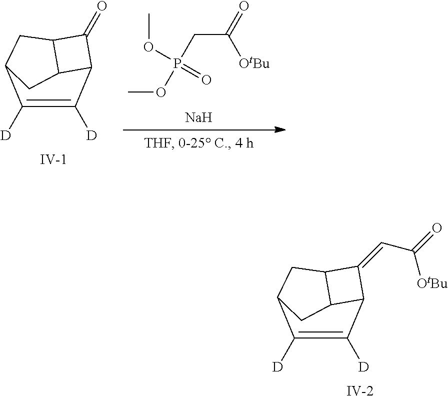

To a solution of tert-butyl 2-dimethoxyphosphorylacetate (1.19 g, 5.29 mmol) in THF (12 mL) was added NaH (264 mg, 6.61 mmol, 60% purity) under N₂ at 0° C. The reaction mixture was stirred for 0.5 hr at 0° C. and then compound IV-1 (600 mg, 4.41 mmol) in THF (12 mL) was added to the mixture. The reaction mixture was stirred for 4 hrs at 25° C. LCMS showed the starting material was consumed, the desired mass was detected. The mixture was quenched by sat. NH₄Cl (20 mL), the organic layer was extracted with EtOAc (30 mL×2), dried over Na₂SO₄, filtered, and concentrated under reduced pressure. The residue was purified by flash silica gel chromatography (12 g Silica Flash Column, Eluent of 0˜2% Ethyl acetate/Commercial hexanes gradient 40 mL/min) to give compound IV-2 (880 mg, 85% yield) as yellow oil.

LC-MS: m/z=179.1, M-56+H⁺

¹H NMR: (400 MHz, CD₃Cl) δ 6.41-6.30 (m, 0.5H), 5.41-5.24 (m, 1H), 3.88-3.64 (m, 1H), 3.38-3.17 (m, 1H), 3.08-2.98 (m, 1H), 2.81-2.68 (m, 1H), 2.03-1.85 (m, 2H), 1.68-1.60 (m, 1H), 1.47 (d, J=6.4 Hz, 9H), 1.37 (dd, J=11.2, 18.4 Hz, 1H).

### Step-10: Preparation of Intermediate IV-3

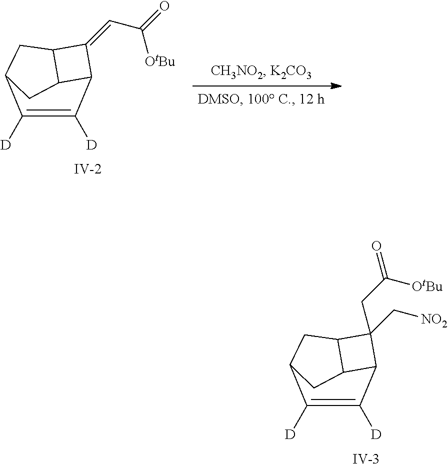

To a solution of compound IV-2 (880 mg, 3.76 mmol) and K₂CO₃ (1.56 g, 11.3 mmol) in DMSO (10 mL) was added CH₃NO₂ (2.29 g, 37.6 mmol) at 25° C. under N₂. The reaction mixture was stirred for 16 hrs at 100° C. LCMS showed the starting material was consumed, the desired mass was detected. The mixture was cooled to 25° C. and diluted with water (30.00 mL), extracted with EtOAc (50.00 mL×2). Then the combined organic phase was washed with brine (20.0 mL), dried over Na₂SO₄, filtered and concentrated. The residue was purified by flash silica gel chromatography (4 g Silica Flash Column, Eluent of 0˜5% Ethyl acetate/Petroleum ether gradient 20 mL/min) to give compound IV-3 (780 mg, 70% yield) as colorless oil.

LC-MS: m/z=318.1, M+23+

¹H NMR: (400 MHz, CD₃CI) 6 6.48 (d, J=7.2 Hz, 1H), 4.98-4.82 (m, 2H), 3.16-3.05 (m, 1H), 2.85-2.79 (m, 1H), 2.71 (d, J=4.8 Hz, 1H), 2.62 (td, J=4.8, 8.8 Hz, 1H), 2.50-2.37 (m, 2H), 1.74-1.60 (m, 2H), 1.53 (td, J=4.0, 11.2 Hz, 1H), 1.45 (s, 9H), 1.29 (d, J=11.2 Hz, 1H).

### Step-11: Preparation of Intermediate IV-4

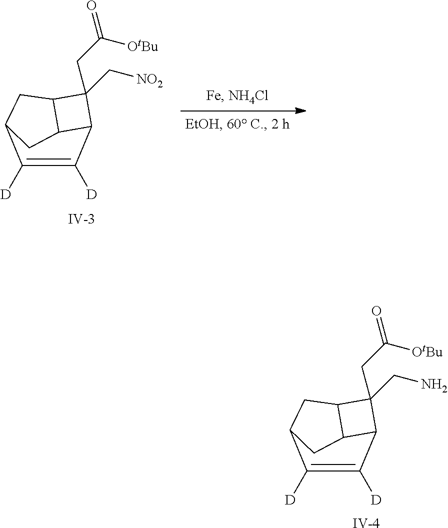

To a solution of compound IV-4 (100 mg, 0.34 mmol) in EtOH (1.6 mL) and H₂O (0.64 mL) was degassed and purged with N₂ for 3 times, and then was slowly added Fe (95 mg, 1.69 mmol) and NH₄Cl (91.0 mg, 1.69 mmol) under N₂ atmosphere at 25° C. Then the mixture was stirred at 80° C. for 2 hrs under N₂ atmosphere. LCMS showed the starting material was consumed, the desired mass was detected. The mixture was filtered, and the filter cake was washed with EtOAc (10 mL). The filtrate was diluted with water (10.00 mL), extracted with EtOAc (20.00 mL×2). Then the combined organic phase was washed with brine (20.0 mL), dried over Na₂SO₄, filtered and concentrated to give compound IV-12b (72 mg, 80% yield) as yellow oil, which was used into next step without purification.

LC-MS: m/z=266.0, M+H⁺

¹H NMR: (400 MHz, CD₃Cl) δ 6.39 (d, J=7.2 Hz, 0.5H), 3.03-2.87 (m, 3H), 2.66 (d, J=4.8 Hz, 1H), 2.52 (dd, J=4.8, 6.4 Hz, 1H), 2.36 (td, J=4.8, 9.2 Hz, 1H), 2.31-2.20 (m, 2H), 1.77 (s, 1H), 1.64-1.55 (m, 1H), 1.51-1.40 (m, 1OH), 1.23 (d, J=11.2 Hz, 1H).

### Step-12: Preparation of Compound IV

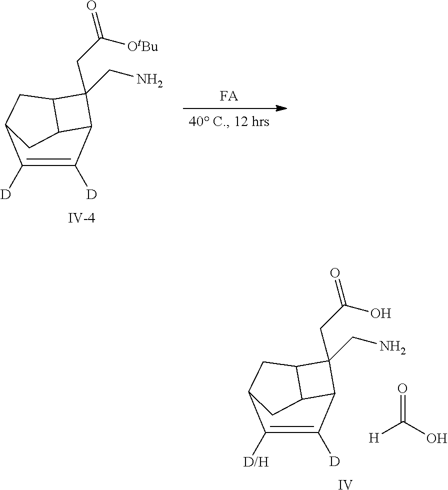

A solution of compound IV-4 (20 mg, 0.08 mmol) in formic acid (0.5 mL) was heated to 40° C. and stirred for 16 hrs. LCMS showed the starting material was consumed, the desired mass was detected. The mixture was concentrated under reduced pressure to give compound IV (12 mg, 76% yield) as yellow gum.

LC-MS: m/z=210.2, M+H⁺

¹H NMR: (400 MHz, D₂O) 6 8.29 (s, 1H), 6.49 (d, J=7.2 Hz, 0.5H), 3.49-3.31 (m, 2H), 3.14-3.00 (m, 1H), 2.68 (d, J=4.4 Hz, 1H), 2.60 (t, J=5.6 Hz, 1H), 2.53-2.37 (m, 3H), 1.68-1.57 (m, 2H), 1.48 (td, J=4.0, 11.2 Hz, 1H), 1.18 (d, J=11.2 Hz, 1H).

### 2. Pharmacological Activity Evaluation

### Example 8: Affinity Assay for α2δ1 and α2δ2 Subunits

Experimental Method: Gabapentin is a known high-affinity ligand for the VGCC α2δ-1 & α2δ-2 subunits. It has been widely used clinically for the treatment of neuropathic pain and epilepsy. Therefore, the in vitro activity of the synthesized compounds was evaluated by measuring their ability to inhibit the binding of [³H] Gabapentin to humanized VGCC α2δ-1 and α2δ-2 subunits. A higher inhibition rate of [³H]Gabapentin binding at a given compound concentration indicates stronger binding affinity of the test compound to the α2δ-1/α2δ-2 subunits, which may correlate with improved in vivo efficacy.

The assay was performed using HEK293 cells expressing recombinant human Cav2.2/β3/α2δ-1 or Cav2.2/β3/α2δ-2 calcium channels. Cell membranes were prepared by standard procedures, and 20 μg of membrane protein was added per well. HEPES buffer (pH 7.4) was used as the assay medium. Each well was incubated with 40 nM [³H]Gabapentin and the test compound at the indicated concentrations for 30 minutes at 25° C.

After incubation, the membranes were harvested by filtration and washed with 50 mM Tris-HCl (pH 7.4). The radioactivity associated with the membranes was measured using liquid scintillation counting to determine the amount of [³H]gabapentin bound.

The Binding (%) was calculated using the formula:

$$= [ ( I - IU ) / ( {I}_{0} - IU \} ] \times 100 \%$$

where:

I is the radioactivity measured when the test compound and [³H]gabapentin is co-incubated with the Cav2.2/β3/α2δ-1 or Cav2.2/β3/α2δ-2 channel membranes.

I₀ is the radioactivity when only [³H]gabapentin is incubated (no test compound).

IU is the radioactivity measured in the presence of 100 μM gabapentin (i.e., non-specific binding control).

The inhibition rate (%) was calculated as:

$$Inhibition ( \% ) = 100 \% - Binding ( \% )$$

The half-maximal inhibitory concentration (IC₅₀) of each compound was determined from its dose-response curve by nonlinear regression using GraphPad Prism 5.10 with a least-squares fit model.

### Experimental Results

TABLE 1
Affinity of Compounds of the Present Disclosure
for α2δ1 and α2δ2 Subunits

| | α2δ1 Binding Assay, | α2δ2 Binding Assay, |
|---|---|---|
| Compound Name | IC₅₀ (nM) | IC₅₀ (nM) |
| Compound I- | 65.31 | 95.47 |
| Peak2 | | |
| Mirogabalin | 39.21 | 74.00 |
| Besilate | | |
| Crisugabalin | 367.70 | 1,085.00 |

Compared to Crisugabalin, the compounds of the present invention exhibit lower half-maximal inhibitory concentrations (IC₅₀) for the inhibition of gabapentin binding to calcium channels, indicating stronger inhibitory activity.

### Example 9: Analgesic Effect of the Disclosed Compounds in the Rat Formalin Model

### 1) Biological Activity Evaluation:

Rats were acclimated to the experimental environment for 3 days prior to testing. Animals weighing between 220-240 g were identified by labeling the base of the tail. A training pad was placed on the laboratory bench, and a white transparent cylindrical chamber was positioned on top of the pad. Thirty minutes before the formal test, a small metal plate was affixed to the plantar surface of the left hind paw of each rat. The rats were then placed individually into the transparent cylinders for habituation.

Test compounds were administered via oral gavage 4 hours in advance (no treatment was given to the sham group). For modeling, 50 μL of 2% formalin was injected subcutaneously into the dorsal surface of the left hind paw (sham group received no formalin injection). Immediately after injection, the animals were placed into an automated locomotor activity monitoring system, which recorded the number of nociceptive behaviors such as paw withdrawal, paw lifting, and paw licking. Behavioral responses were automatically recorded and analyzed over a 0 to 60-minute period following formalin administration.

### 2) Evaluation Parameters:

Following subcutaneous injection of 50 μL of 2% formalin, spontaneous locomotor activity (including lifting, licking, and flinching of the injected paw) was recorded and analyzed over a period of 0-60 minutes. This time-window was divided into two distinct phases for evaluation:
Phase 1 (0-9 minutes): Early acute nociceptive phase
Phase II (10-60 minutes): Late inflammatory or tonic phase

The cumulative number of paw lifts was used as a primary indicator of nociceptive behavior.

The Maximum Possible Effect (MPE %) was calculated to quantify the analgesic efficacy of the test compound using the following formula:

$$MPE \% = [ \text{} ( Vehicle - sham ) - ( Drug froup - Sham ) ] \text{} / ( Vehicle - sham ) \times 100 \%$$

### Notes:

a) The Sham group is defined as 100% MPE, while the Vehicle (model control) group is defined as 0% MPE_{∘}
b) Background interference was minimized by subtracting the Sham group's spontaneous activity counts from each group's corresponding values

### 3) Data Acquisition and Statistical Analysis:

All data were expressed as mean±standard error of the mean (SEM). Statistical analysis was conducted using GraphPad Prism version 8.0.1. A one-way ANOVA followed by Dunnett's multiple comparisons test was used to assess statistical differences between treatment and control groups. A p-value <0.05 was considered statistically significant.

The in vivo pharmacological effects of the compounds disclosed in this study are summarized in Table 2.

TABLE 2
In Vivo Pharmacological Effects of the Disclosed Compounds
in the Formalin-Induced Pain Model in Rats.

| | | Maximum Possible | |
|---|---|---|---|
| | Dosage | Analgesic Effect | ED₅₀ |
| Compound Name | (mg/kg) | (MPE %) | (mg/kg) |
| Compound I- | 3 | 31% | 6.6 |
| Peak2 | 10 | 57% | |
| | 30 | 89% | |
| Compound III - | 10 | 39% | — |
| Peak2 | | | |
| Mirogabalin | 3 | 31% | 8.3 |
| Besilate | 10 | 52% | |
| | 30 | 81% | |
| Crisugabalin | 10 | 19% | 17.3 |
| | 30 | 41% | |
| | 100 | 57% | |

Compared to Crisugabalin, the compounds disclosed in this invention significantly suppress formalin-induced hyperalgesia in the rat formalin model. The compounds exhibit a lower median effective dose (ED₅₀), indicating stronger analgesic activity.

### Example 10: In-Vivo Pharmacokinetic/Pharmacodynamic Data

Experimental Procedure: Male Sprague-Dawley (SD) rats were fasted for 12 hours prior to the experiment but allowed free access to water. Compound I of this invention, Crisugabalin, and Mirogabalin Besilate were each dissolved in a solvent mixture of 5% DMSO+10% Solutol+85% saline & then administered either orally or intravenously to male SD rats, with three rats in each group.

Blood samples were collected via the jugular vein at 5 min, 15 min, 30 min, 1 h, 2 h, 4 h, 6 h, 8 h, and 24 h post-dosing. Approximately 0.2 mL of blood was collected for each sample using EDTA-K₂ as an anticoagulant. After collection, samples were immediately placed on wet ice and centrifuged within one hour to separate the plasma (centrifugation conditions: 6000 g, 3 minutes, 2-8° C.). Plasma samples were stored at −80° C. until analysis.

Drug concentrations of the prototype compound in plasma were determined using a validated HPLC-ESI-MS method. Based on plasma drug concentration data at various time points, pharmacokinetic parameters were calculated using Phoenix WinNonlin 8.2.0. The parameters included AUC₀−t, AUC₀−∞, MRT₀−∞, Cmax, Tmax, and T½, along with their corresponding means and standard deviations.

The test results are presented in Table 3.

TABLE 3
Pharmacokinetic Data of the Disclosed Compounds

| | Dose | Tlast | T_{1/2} | Tₘₐₓ | Cₘₐₓ | AUC₍₀₋ₜ₎ | AUC_{(0-∞)} | Cl | F |
|---|---|---|---|---|---|---|---|---|---|
| Compd. | (mg/kg) | (h) | (h) | (h) | (ng/mL) | (h*ng/mL) | (h*ng/mL) | (mL/min/kg) | (%) |
| Compound | 3.0 IV. | 13.3 | 1.12 | 0.0830 | 3147 | 3172 | 3179 | 16.0 | — |
| I-Peak2 | | | | | | | | | |
| | 1.0 PO. | 13.3 | 1.39 | 2.00 | 318 | 1153 | 1179 | — | 111.23 |
| | 3.0 PO. | 13.3 | 1.65 | 0.417 | 1105 | 3172 | 3194 | — | 100.46 |
| | 10.0 PO. | 24.0 | 3.40 | 0.417 | 3267 | 10664 | 10734 | — | 101.28 |
| Mirogabalin | 3.0 IV. | 8.0 | 0.94 | 0.0833 | 3680 | 3800 | 3811 | 13.51 | — |
| Besylate | 1.0 PO. | 8.0 | 0.96 | 1.17 | 254 | 1029 | 1043 | — | 82.1 |
| | 3.0 PO. | 18.7 | 2.40 | 0.67 | 837 | 2915 | 2927 | — | 76.8 |
| | 10.0 PO. | 18.7 | 2.51 | 1.50 | 2537 | 10075 | 10139 | — | 79.8 |
| Crisugabalin | 3.0 IV. | 24.0 | 3.28 | 0.0833 | 3360 | 6964 | 6982 | 7.36 | — |
| | 10.0 PO. | 24.0 | 3.09 | 1.33 | 3817 | 16479 | 16531 | — | 71.0 |
| | 30.0 PO. | 24.0 | 2.54 | 1.00 | 8197 | 45922 | 46887 | — | 67.2 |
| | 100.0 PO. | 24.0 | 4.22 | 3.00 | 23767 | 146150 | 149530 | — | 64.3 |

### Experimental Conclusion

From the above data, it is evident that the representative compound of the present invention, Compound I-peak2, is rapidly absorbed after oral administration and demonstrates very high bioavailability, making it well-suited for oral administration.

Embodiment 1: A compound of formula (I), its stereoisomers, or a pharmaceutically acceptable salt thereof,

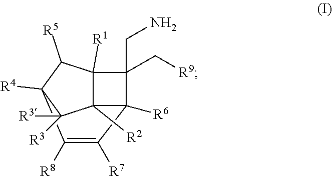

R1, R2, R3, R3′, R4, R5, R6, R7, and R8 are each independently selected from hydrogen, deuterium, halogen, substituted or unsubstituted C1-C6 alkyl, and substituted or unsubstituted C3-C6 cycloalkyl, wherein the substituted C1-C6 alkyl and substituted C3-C6 cycloalkyl are optionally further substituted with 0 to 6 substituents selected from halogen, hydroxyl, carboxyl, amino, cyano, C1-C3 alkyl, and C3-C6 cycloalkyl; and
R9 is selected from carboxylic acid (—COOH),

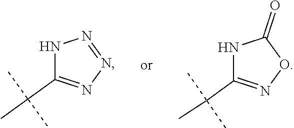

Embodiment 2: The compound according to embodiment 1, and its stereoisomers, or a pharmaceutically acceptable salt thereof, wherein:
R1, R2, R3, R3′, R4, R5, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl; and
R9 is selected from carboxylic acid (—COOH),

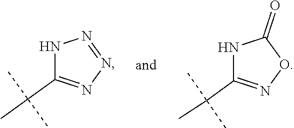

Embodiment 3: The compound of formula (II) according to embodiment 1, its stereoisomers, or a pharmaceutically acceptable salt thereof,

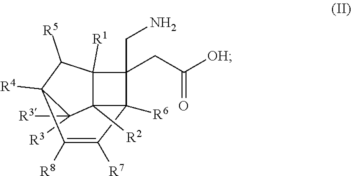

R1 and R5 are each independently selected from hydrogen, deuterium, methyl, ethyl, propyl, isopropyl, butyl, tert-butyl, and substituted C1-C6 alkyl, wherein the substituted C1-C6 alkyl is optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl;
R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl; and
R4, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

Embodiment 4: The compound of formula (II-1) according to embodiment 3, its stereoisomers, or a pharmaceutically acceptable salt thereof,

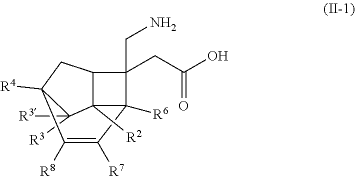

R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl and
R4, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

Embodiment 5: The compound of formula (III) or (IV) according to embodiment 1, its stereoisomers, or a pharmaceutically acceptable salt thereof.

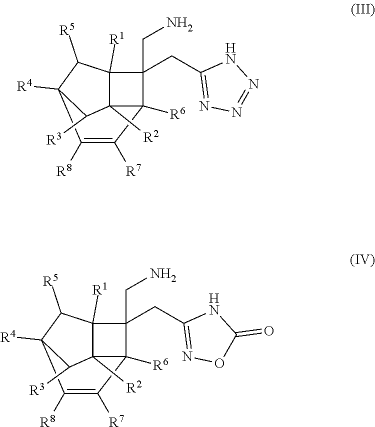

R1 and R5 are each independently selected from hydrogen, deuterium, methyl, ethyl, propyl, isopropyl, butyl, tert-butyl, and substituted C1-C6 alkyl, wherein the substituted C1-C6 alkyl is optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl;
R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl; and
R4, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

Embodiment 6: The compound according to embodiment 1, wherein the compound is selected from the group consisting of the following compounds, or any stereoisomer or pharmaceutically acceptable salt thereof,

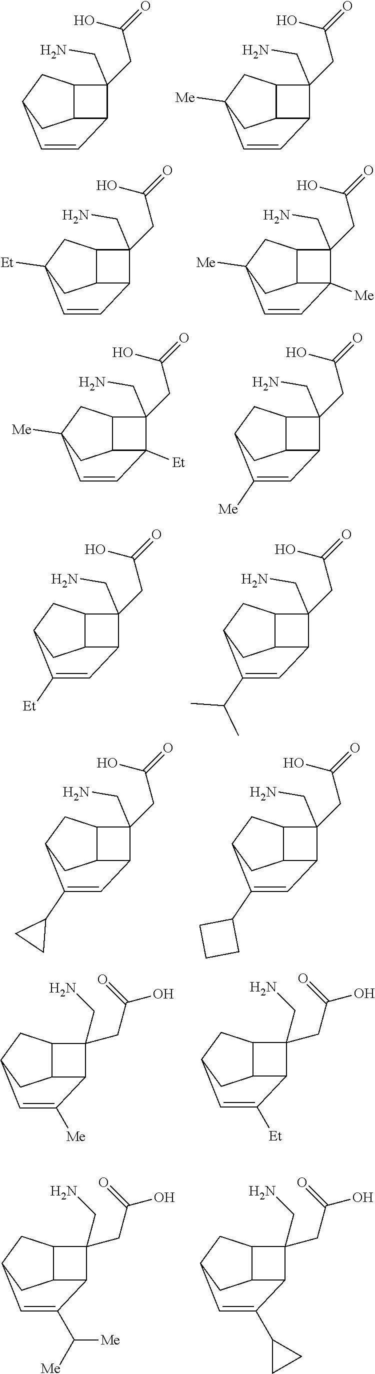

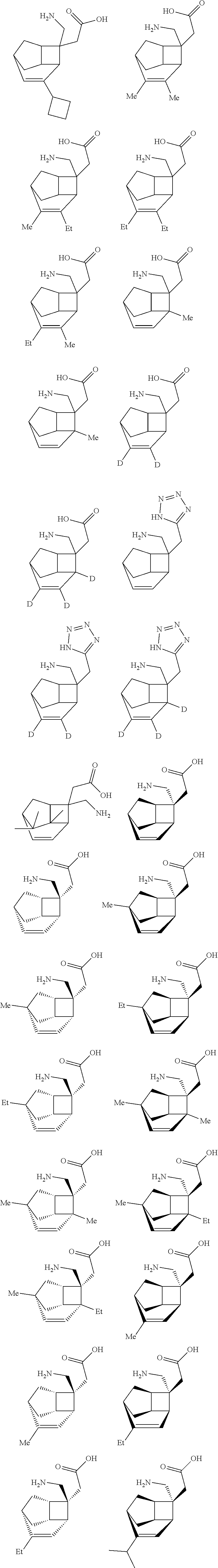

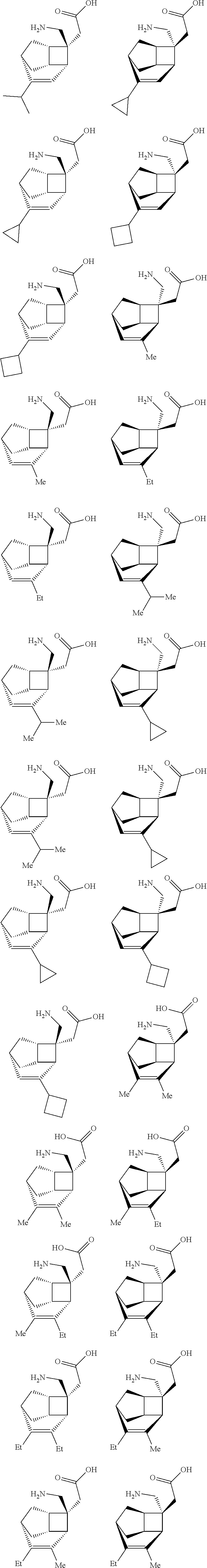

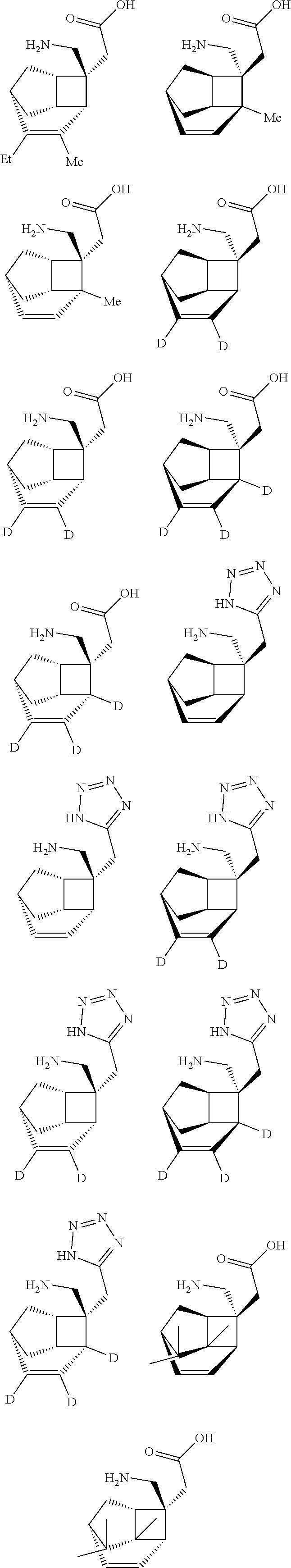

Embodiment 7: A pharmaceutical composition comprising a compound of formula (I) as defined in embodiment 1, or a stereoisomer, solvate, metabolite, prodrug, pharmaceutically acceptable salt, or cocrystal thereof, and one or more pharmaceutically acceptable carriers and/or excipients.

Embodiment 8: A method of treating and/or preventing diseases or disorders associated with ligands of the α2δ subunit of voltage-gated calcium channels, the method comprising administering a therapeutically effective dose of the compound of embodiment 1 to a subject in need thereof.

Embodiment 9: A method of treating pain mediated by the α2δ subunit of a voltage-gated calcium channel in a subject in need thereof, the method comprising administering to the subject a therapeutically effective amount of the compound of embodiment 1.

Embodiment 10: The method according to embodiment 9, wherein the pain comprises postherpetic neuralgia, trigeminal neuralgia, migraine, pain associated with osteoarthritis or rheumatoid arthritis, low back pain, sciatica, toothache, burn-induced pain, pain caused by diabetic neuropathy, diabetic peripheral neuropathic pain, diabetic peripheral neuropathy, diabetic peripheral neuralgia, chemotherapy-induced neuropathic pain, neurological diseases, central neuropathic pain, central nervous system-related pain, peripheral nervous system disorders, peripheral neuralgia, HIV-related neuralgia, AIDS-related neuralgia, cancer-related neuralgia or non-neuropathic pain, acute or chronic tension headache, postoperative pain, fibromyalgia, epilepsy, generalized anxiety disorder, or restless legs syndrome, chronic kidney disease, renal insufficiency, or perioperative orthopedic analgesia.

Embodiment 11: The method of embodiment 10, wherein the pain is peripheral neuropathic pain.

Embodiment 12: The method of embodiment 10, wherein the pain is diabetic peripheral neuropathy or postherpetic neuralgia.

Embodiment 13: An intermediate compound, its stereoisomer, or a pharmaceutically acceptable salt thereof, for the preparation of the compound of general formula (I) as defined in embodiment 1, wherein:

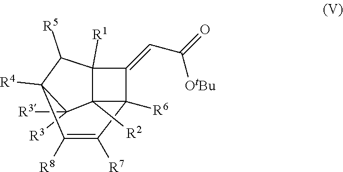

R1, R2, R3, R3′, R4, R5, R6, R7, and R8 are each independently selected from hydrogen, deuterium, halogen, substituted or unsubstituted C1-C6 alkyl, and substituted or unsubstituted C3-C6 cycloalkyl, wherein the substituted C1-C6 alkyl and substituted C3-C6 cycloalkyl are optionally further substituted with 0 to 6 substituents selected from halogen, hydroxyl, carboxyl, amino, cyano, C1-C3 alkyl, and C3-C6 cycloalkyl;

Embodiment 14: The compound according to embodiment 13, or a stereoisomer or pharmaceutically acceptable salt thereof, wherein the compound is selected from those of formula (V-1).

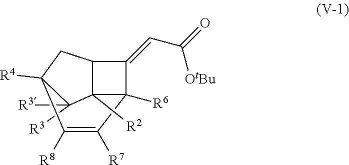

R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl and
R4, R6, R7, and R8 are each independently selected from hydrogen, deuterium, fluorine, chlorine, bromine, iodine, methyl, ethyl, propyl, isopropyl, butyl, t-butyl, cyclopropyl, cyclobutyl, cyclopentyl, cyclohexyl, substituted C1-C6 alkyl, & substituted C3-C6 cycloalkyl, wherein the substituted C1-C6 alkyl & substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, & cyclohexy.

Embodiment 15: The compound according to embodiment 13, or a stereoisomer or pharmaceutically acceptable salt thereof, wherein the compound is selected from one of the following structures:

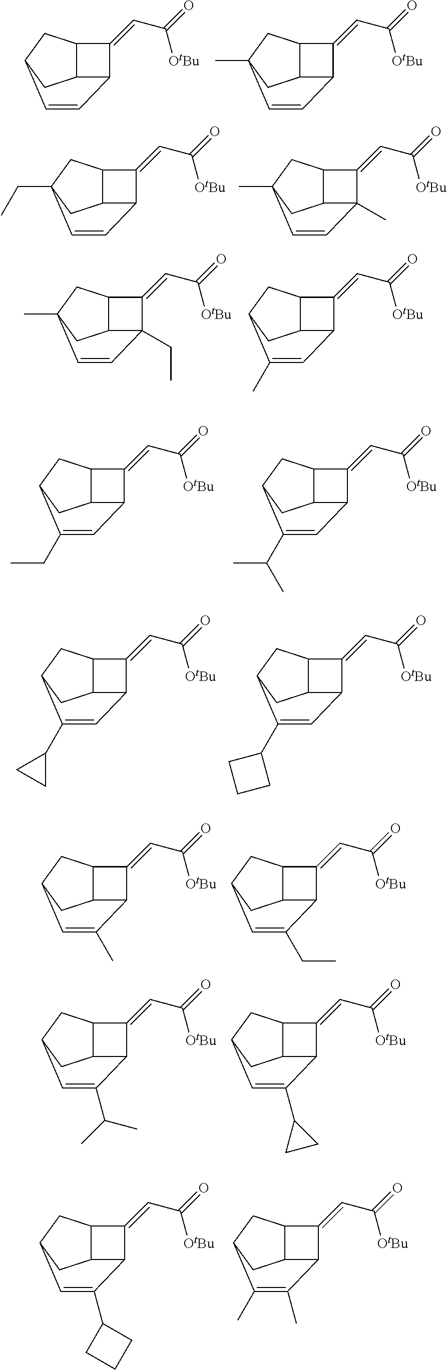

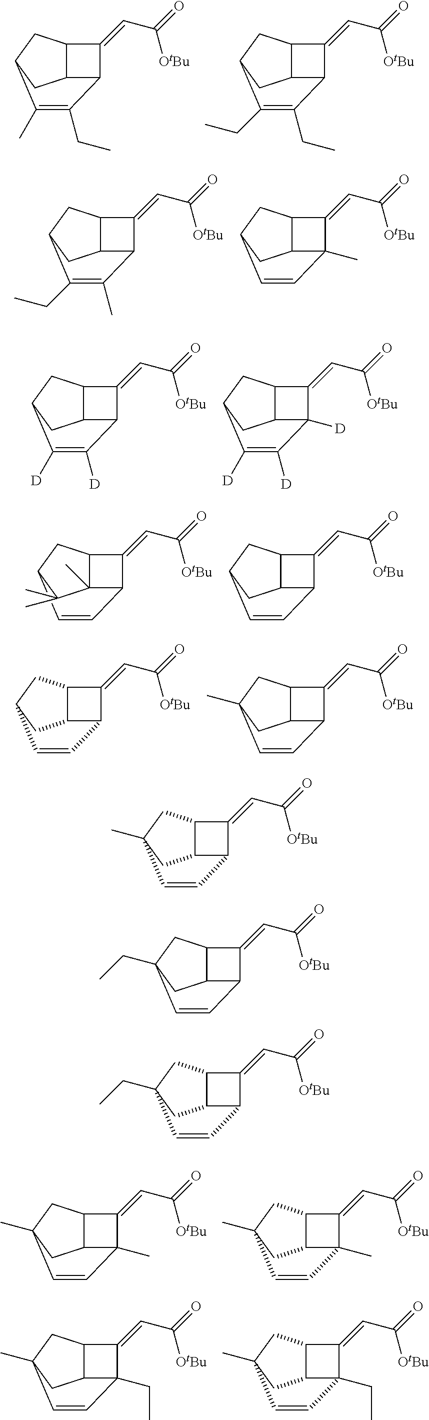

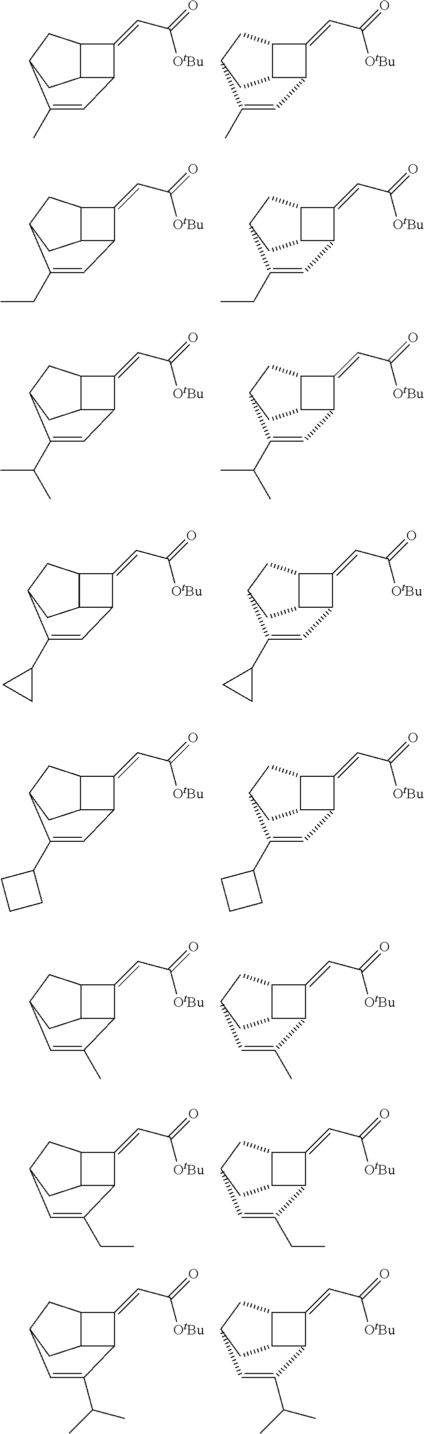

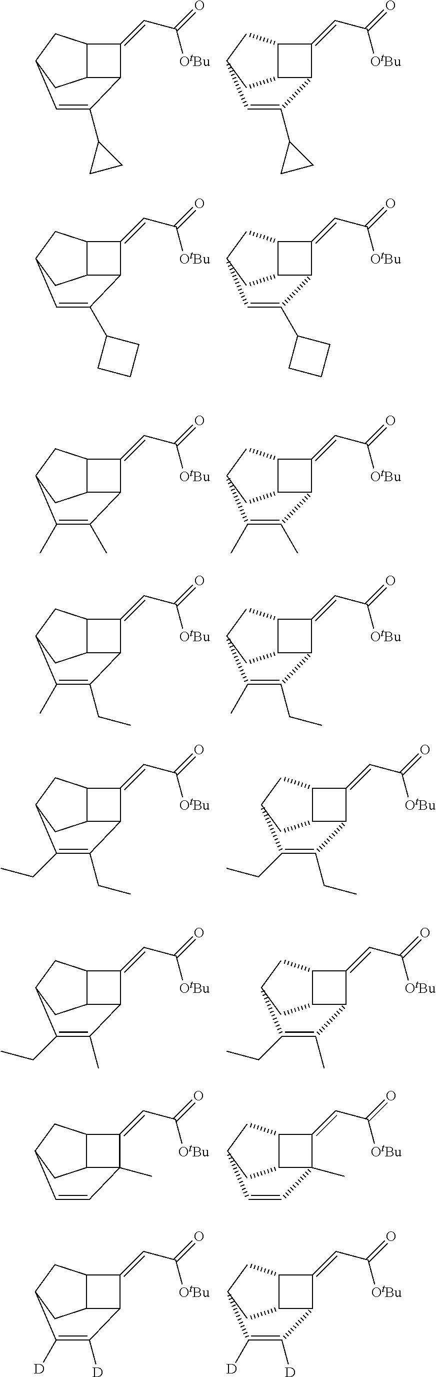

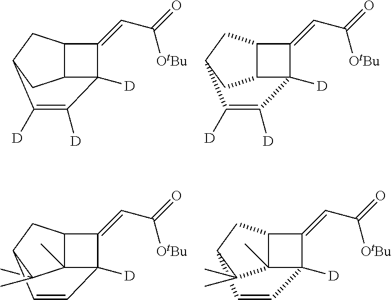

1. A compound of formula (I), its stereoisomers, or a pharmaceutically acceptable salt thereof, 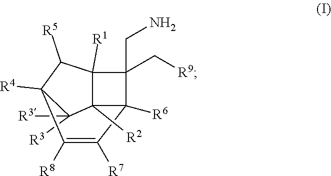
R1, R2, R3, R3′, R4, R5, R6, R7, and R8 are each independently selected from hydrogen, deuterium, halogen, substituted or unsubstituted C1-C6 alkyl, and substituted or unsubstituted C3-C6 cycloalkyl, wherein the substituted C1-C6 alkyl and substituted C3-C6 cycloalkyl are optionally further substituted with 0 to 6 substituents selected from halogen, hydroxyl, carboxyl, amino, cyano, C1-C3 alkyl, and C3-C6 cycloalkyl; and
R9 is selected from carboxylic acid (—COOH),

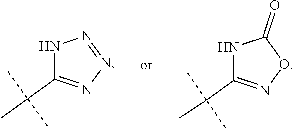

2. The compound according to claim 1, and its stereoisomers, or a pharmaceutically acceptable salt thereof, wherein:
R1, R2, R3, R3′, R4, R5, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl; and
R9 is selected from carboxylic acid (—COOH),

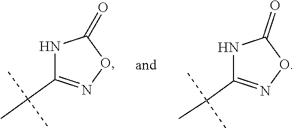

3. The compound of formula (II) according to claim 1, its stereoisomers, or a pharmaceutically acceptable salt thereof, 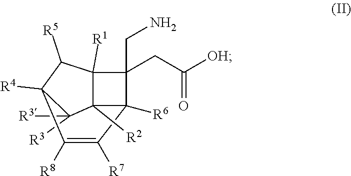
R1 and R5 are each independently selected from hydrogen, deuterium, methyl, ethyl, propyl, isopropyl, butyl, tert-butyl, and substituted C1-C6 alkyl, wherein the substituted C1-C6 alkyl is optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl;
R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl; and
R4, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

4. The compound of formula (II-1) according to claim 3, its stereoisomers, or a pharmaceutically acceptable salt thereof, 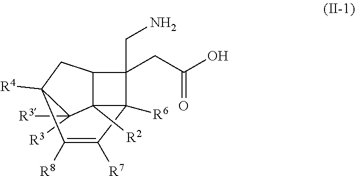
R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl; and
R4, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

5. The compound of formula (III) or (IV) according to claim 1, its stereoisomers, or a pharmaceutically acceptable salt thereof. 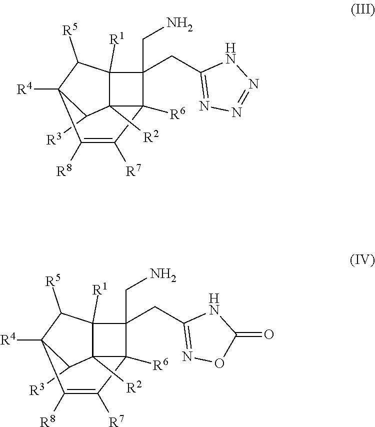
R1 and R5 are each independently selected from hydrogen, deuterium, methyl, ethyl, propyl, isopropyl, butyl, tert-butyl, and substituted C1-C6 alkyl, wherein the substituted C1-C6 alkyl is optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl;
R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl; and
R4, R6, R7, and R8 are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl.

6. The compound according to claim 1, wherein the compound is selected from the group consisting of the following compounds, or any stereoisomer or pharmaceutically acceptable salt thereof,

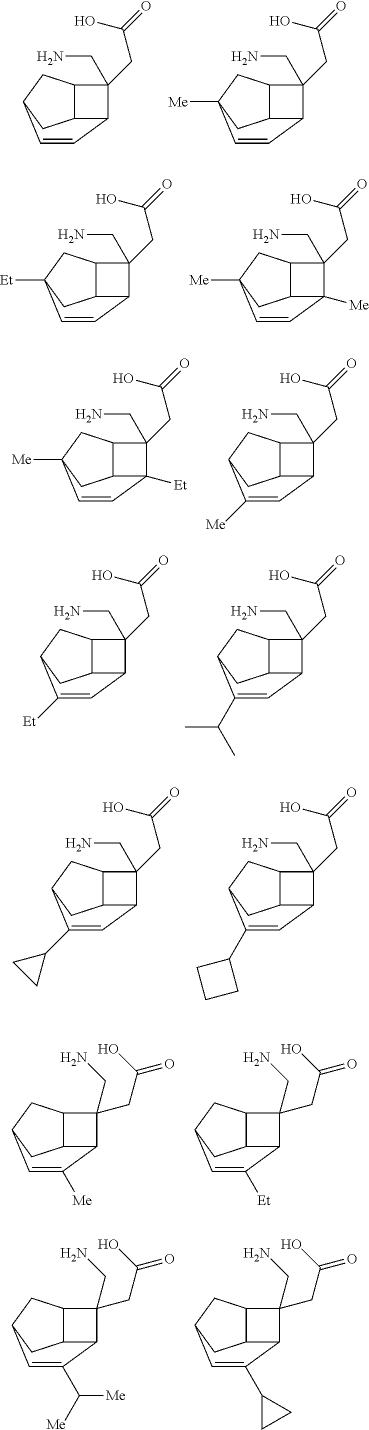

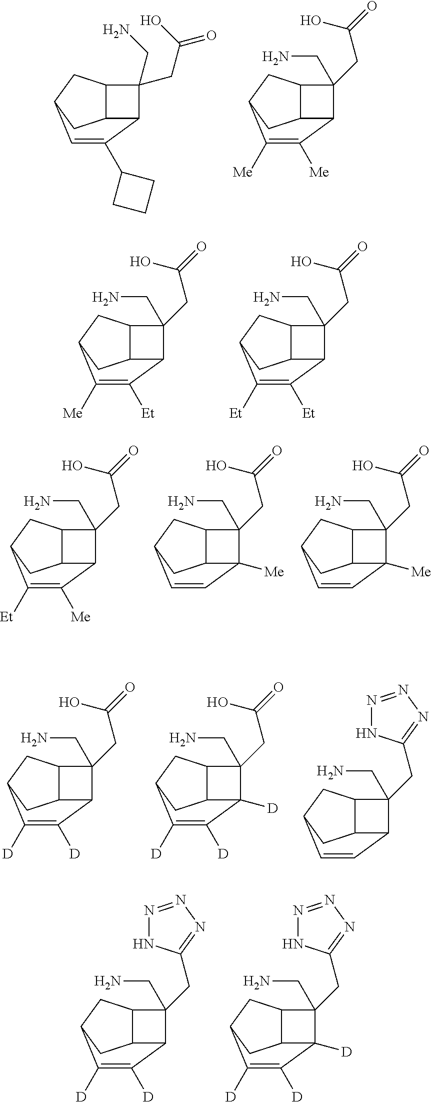

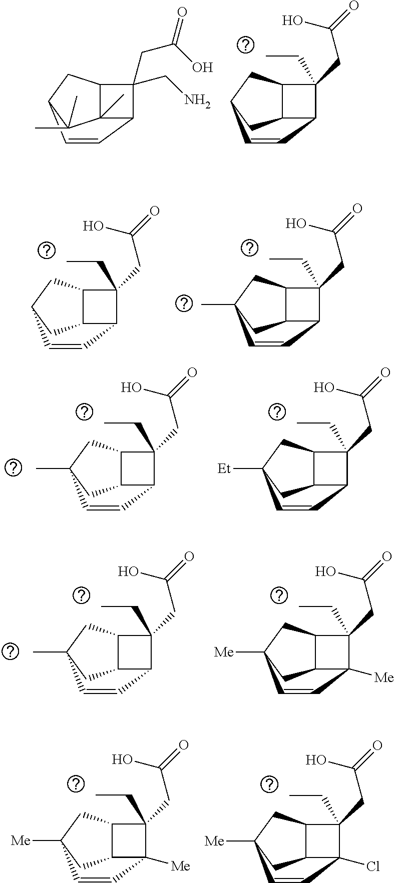

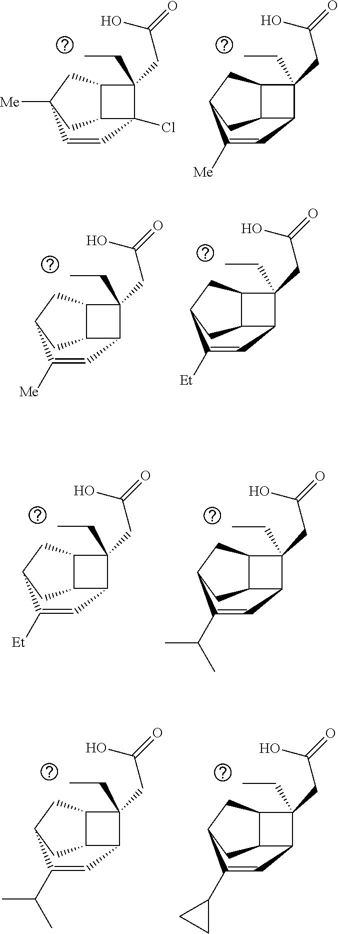

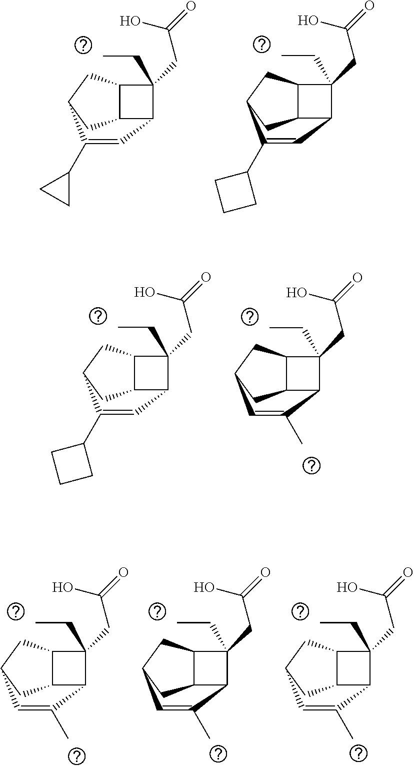

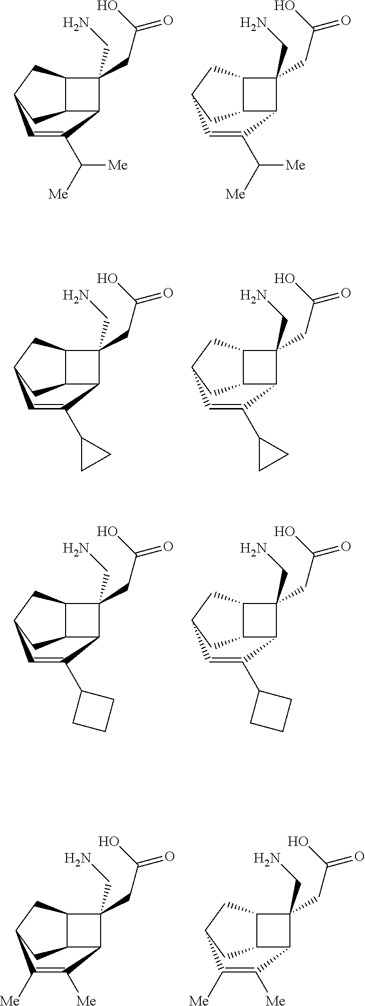

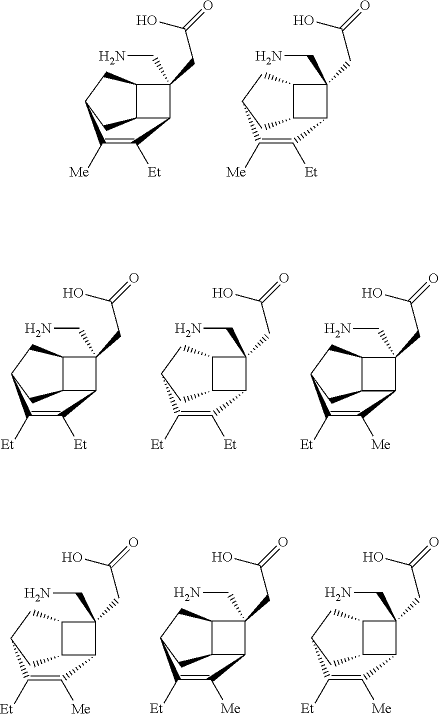

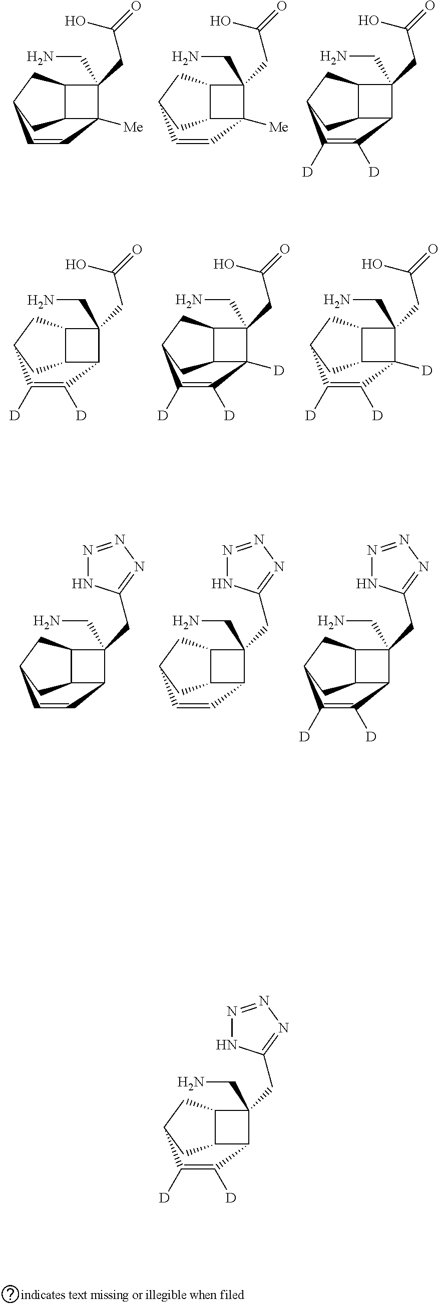

7. A pharmaceutical composition comprising a compound of formula (I) as defined in claim 1, or a stereoisomer, solvate, metabolite, prodrug, pharmaceutically acceptable salt, or cocrystal thereof, and one or more pharmaceutically acceptable carriers and/or excipients.

8. A method of treating and/or preventing diseases or disorders associated with ligands of the α2δ subunit of voltage-gated calcium channels, the method comprising administering a therapeutically effective dose of the compound of claim 1 to a subject in need thereof.

9. A method of treating pain mediated by the α2δ subunit of a voltage-gated calcium channel in a subject in need thereof, the method comprising administering to the subject a therapeutically effective amount of the compound of claim 1.

10. The method according to claim 9, wherein the pain comprises postherpetic neuralgia, trigeminal neuralgia, migraine, pain associated with osteoarthritis or rheumatoid arthritis, low back pain, sciatica, toothache, burn-induced pain, pain caused by diabetic neuropathy, diabetic peripheral neuropathic pain, diabetic peripheral neuropathy, diabetic peripheral neuralgia, chemotherapy-induced neuropathic pain, neurological diseases, central neuropathic pain, central nervous system-related pain, peripheral nervous system disorders, peripheral neuralgia, HIV-related neuralgia, AIDS-related neuralgia, cancer-related neuralgia or non-neuropathic pain, acute or chronic tension headache, postoperative pain, fibromyalgia, epilepsy, generalized anxiety disorder, or restless legs syndrome, chronic kidney disease, renal insufficiency, or perioperative orthopedic analgesia.

11. The method of claim 10, wherein the pain is peripheral neuropathic pain.

12. The method of claim 10, wherein the pain is diabetic peripheral neuropathy or postherpetic neuralgia.

13. An intermediate compound, its stereoisomer, or a pharmaceutically acceptable salt thereof, for the preparation of the compound of general formula (I) as defined in claim 1, wherein: 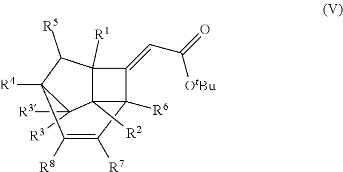
R1, R2, R3, R3′, R4, R5, R6, R7, and R8 are each independently selected from hydrogen, deuterium, halogen, substituted or unsubstituted C1-C6 alkyl, and substituted or unsubstituted C3-C6 cycloalkyl, wherein the substituted C1-C6 alkyl and substituted C3-C6 cycloalkyl are optionally further substituted with 0 to 6 substituents selected from halogen, hydroxyl, carboxyl, amino, cyano, C1-C3 alkyl, and C3-C6 cycloalkyl.

14. The compound according to claim 13, or a stereoisomer or pharmaceutically acceptable salt thereof, wherein the compound is selected from those of formula (V-1). 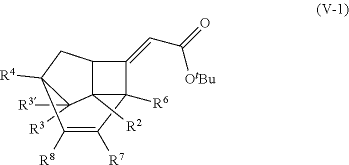
R2, R3, and R3′ are each independently selected from hydrogen, deuterium, fluorine, chlorine, bromine, iodine, methyl, ethyl, propyl, isopropyl, butyl, tert-butyl, cyclopropyl, cyclobutyl, cyclopentyl, cyclohexyl, substituted C1-C6 alkyl, and substituted C3-C6 cycloalkyl, wherein the substituted C1-C6 alkyl and substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, and cyclohexyl; and
R4, R6, R7, and R8 are each independently selected from hydrogen, deuterium, fluorine, chlorine, bromine, iodine, methyl, ethyl, propyl, isopropyl, butyl, t-butyl, cyclopropyl, cyclobutyl, cyclopentyl, cyclohexyl, substituted C1-C6 alkyl, & substituted C3-C6 cycloalkyl, wherein the substituted C1-C6 alkyl & substituted C3-C6 cycloalkyl are optionally further substituted with 0 to 6 substituents selected from deuterium, fluorine, chlorine, bromine, iodine, hydroxyl, carboxyl, amino, cyano, methyl, ethyl, propyl, isopropyl, cyclopropyl, cyclobutyl, cyclopentyl, & cyclohexy.

15. The compound according to claim 13, or a stereoisomer or pharmaceutically acceptable salt thereof, wherein the compound is selected from one of the following structures 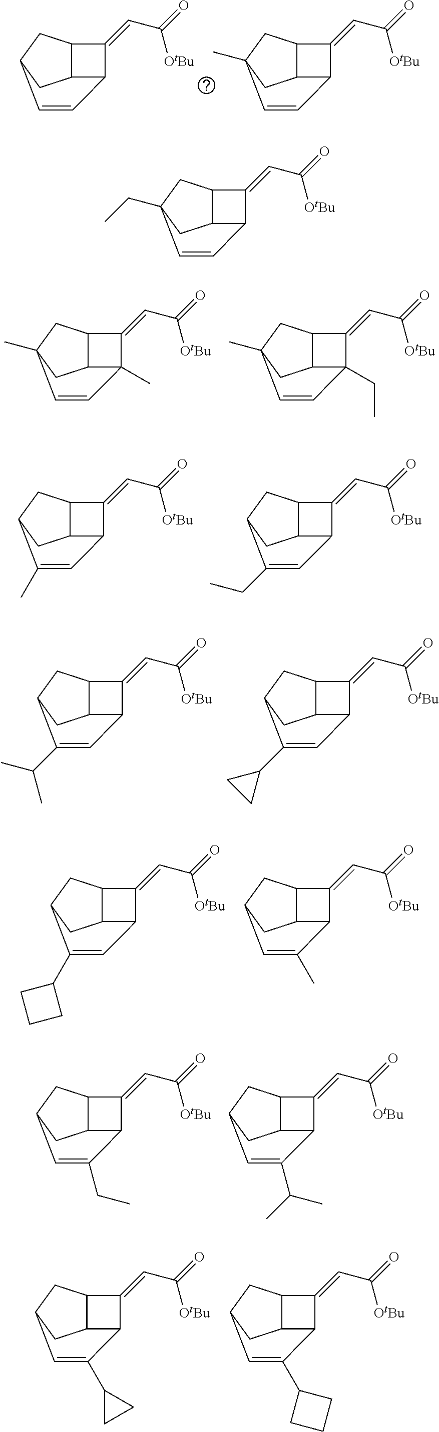 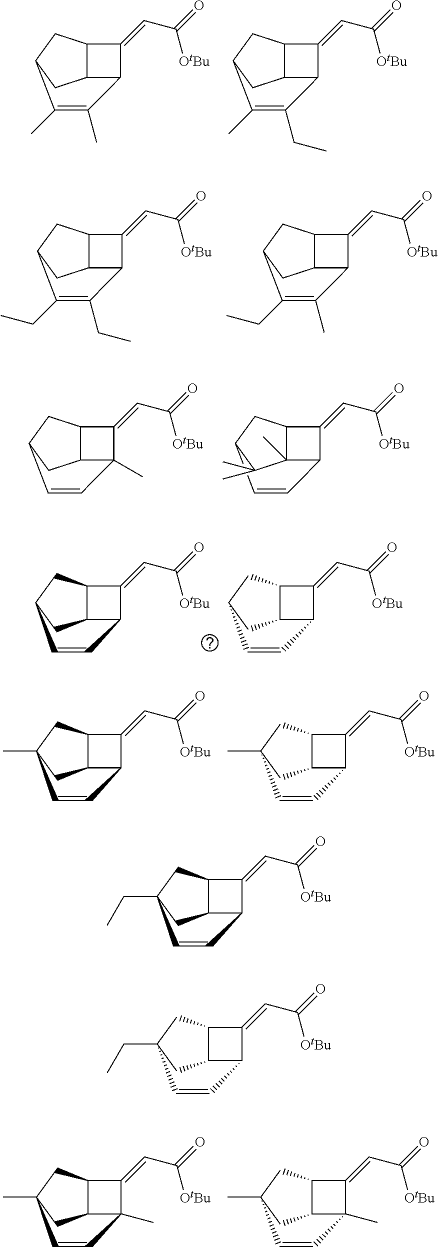 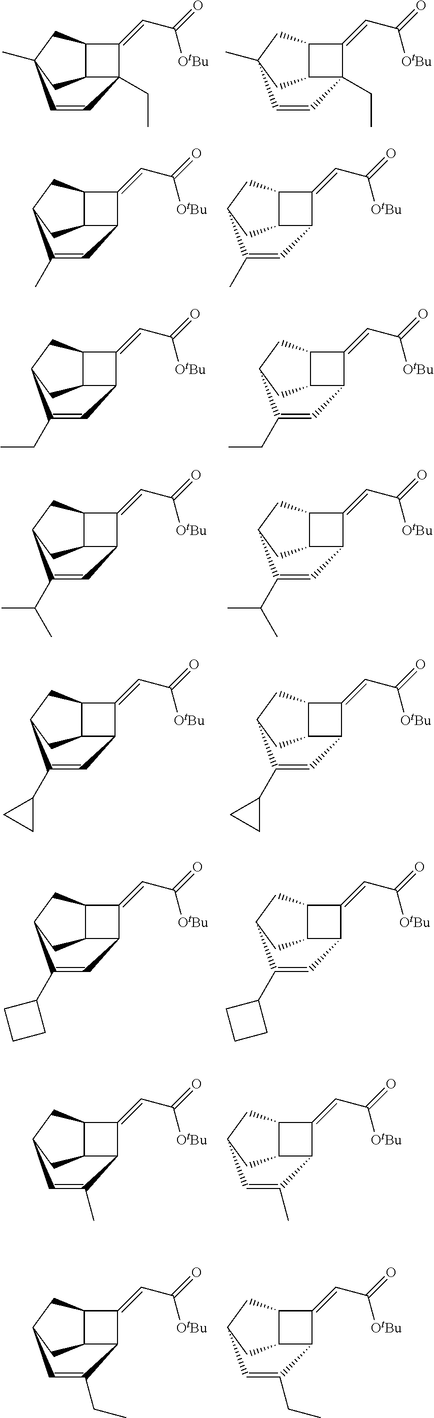 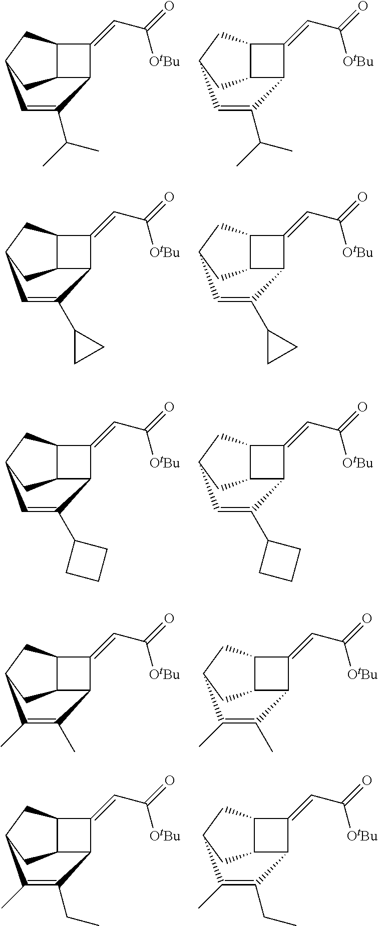 [chemical structure]
